(19) United States
(12) Patent Application Publication
(10) Pub. No.: US 2025/0361262 A1
(43) Pub. Date: Nov. 27, 2025

(54) 3'-BLOCKED NUCLEOTIDES, METHODS OF DEBLOCKING THE SAME, AND METHODS OF SYNTHESIZING POLYNUCLEOTIDES USING THE SAME

(71) Applicant: Illumina, Inc., San Diego, CA (US)

(72) Inventors: Jeffrey Mandell, San Diego, CA (US); Yin Nah Teo, Singapore (SG); Daniel Lukamto, Singapore (SG); Xiangyuan Yang, Singapore (SG); Jean-Alexandre Richard, Singapore (SG); Sherman Lauw, Singapore (SG); Hamed Ghomi, Cambridge (GB); Xiaolin Wu, Cambridge (GB); Wayne George, Cambridge (GB)

(73) Assignee: Illumina, Inc., San Diego, CA (US)

(21) Appl. No.: 18/869,195

(22) PCT Filed: May 16, 2023
(86) PCT No.: PCT/US2023/022435

§ 371 Date: Nov. 25, 2024

Related U.S. Application Data
(60) Provisional application No. 63/346,750, filed on May 27, 2022.

(51) Int. Cl. C07H 21/00; C07H 19/10; C12Q 1/6869

(52) U.S. Cl. CPC C07H 21/00; C07H 19/10; C12Q 1/6869

(57) ABSTRACT

3′-blocked nucleotides, methods of deblocking the same, and methods of synthesizing polynucleotides using the same are provided herein. In some examples, a nucleotide is disposed within the aperture on the first side of a nanopore. The nucleotide may be coupled to a 3′-blocking group including a trigger. The trigger may be selectively activated using an initiator. The activated trigger may be used to remove the 3′-blocking group from the nucleotide.

### CROSS-REFERENCE TO RELATED APPLICATION

This application claims the benefit of U.S. Provisional Patent Application No. 63/346,750, filed May 27, 2022 and entitled “3′-BLOCKED NUCLEOTIDES, METHODS OF DEBLOCKING THE SAME, AND METHODS OF SYNTHESIZING POLYNUCLEOTIDES USING THE SAME,” the entire contents of which are incorporated by reference herein.

### BACKGROUND

A significant amount of academic and corporate time and energy has been invested into using nanopores to sequence polynucleotides. For example, the dwell time has been measured for complexes of DNA with the Klenow fragment (KF) of DNA polymerase I atop a nanopore in an applied electric field. Or, for example, a current or flux-measuring sensor has been used in experiments involving DNA captured in an α-hemolysin nanopore. Or, for example, KF-DNA complexes have been distinguished on the basis of their properties when captured in an electric field atop an α-hemolysin nanopore. In still another example, polynucleotide sequencing is performed using a single polymerase enzyme complex including a polymerase enzyme and a template nucleic acid attached proximal to a nanopore, and nucleotide analogs in solution. The nucleotide analogs include charge blockade labels that are attached to the polyphosphate portion of the nucleotide analog such that the charge blockade labels are cleaved when the nucleotide analog is incorporated into a polynucleotide that is being synthesized. The charge blockade label is detected by the nanopore to determine the presence and identity of the incorporated nucleotide and thereby determine the sequence of a template polynucleotide. In still other examples, constructs include a transmembrane protein nanopore subunit and a nucleic acid handling enzyme.

However, such previously known compositions, systems, and methods may not necessarily be sufficiently robust, reproducible, or sensitive and may not have sufficiently high throughput for practical implementation, e.g., demanding commercial applications such as genome sequencing in clinical and other settings that demand cost effective and highly accurate operation. Accordingly, what is needed are improved compositions, systems, and methods for sequencing polynucleotides, which may include synthesizing polynucleotides.

### SUMMARY

3′-blocked nucleotides, methods of deblocking the same, and methods of synthesizing polynucleotides using the same are provided herein.

Some examples herein provide a method of deblocking a nucleotide using a nanopore. The nanopore may include a first side and a second side, an aperture extending through the first and second sides. The method may include disposing a nucleotide within the aperture on the first side of the nanopore. The nucleotide may be coupled to a 3′-blocking group including a trigger. The method may include selectively activating the trigger using an initiator. The method may include using the activated trigger to remove 3′-blocking group from the nucleotide.

In some examples, the initiator is located on the second side of the nanopore and substantially not located on the first side of the nanopore. In some examples, the initiator is only located on the second side of the nanopore. In some examples, the initiator is located inside of the aperture. In some examples, the initiator is within a fluid in contact with the second side of the nanopore. In some examples, the initiator is coupled to the second side of the nanopore. In some examples, the initiator includes a selenocysteine group.

In some examples, removing 3′-blocking group provides the nucleotide with a 3′-OH group. In some examples, removing 3′-blocking group provides the nucleotide with a 3′-NH₂ group.

In some examples, the initiator includes a reducing agent. In some examples, the reducing agent is selected from the group consisting of glutathione (GSH), seleno-glutathione (GSeH), selenoenzyme thioredoxin, NADP/NADPH, dithiothreitol (DTT) and modifications of the same, cyclodithiothreitol (cDTT), tris(hydroxypropyl)phosphine, and tris(2-carboxyethyl)phosphine (TCEP).

In some examples, 3′-blocking group includes a disulfide bond. In some examples, activating the trigger includes reducing the disulfide bond. In some examples, the 3′-blocking group has the structure:

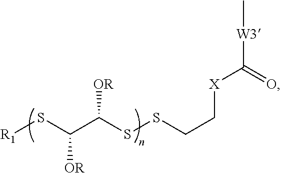

where n is at least one, W is O or NH, X is O or N, R is H, SO³⁻, or PO₃²⁻, and R₁ is selected from the group consisting of

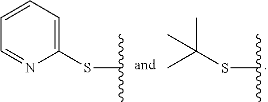

In some examples, 3′-blocking group has the structure:

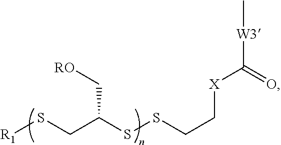

where n is at least one, W is O or NH, X is O or N, R is H, SO³⁻, or PO₃²⁻, and R₁ is selected from the group consisting of

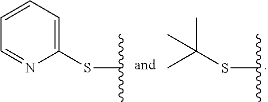

In some examples, 3′-blocking group has the structure:

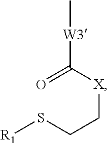

where W is O or NH, X is O or N, and R₁ is selected from the group consisting of

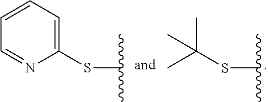

In some examples, the trigger is located on the second side of the nanopore when it is activated.

In some examples, 3′-blocking group further includes an elongated body including a first end coupled to the nucleotide, a second end, and the trigger. In some examples, removing 3′-blocking group includes degrading the elongated body. In some examples, the 3′-blocking group includes one or more monomers. In some examples, the elongated body includes a plurality of the monomers, and wherein degrading the elongated body of the 3′-blocking group includes cascading cyclizations of the monomers.

In some examples, the one or more monomers are selected from the group consisting of:

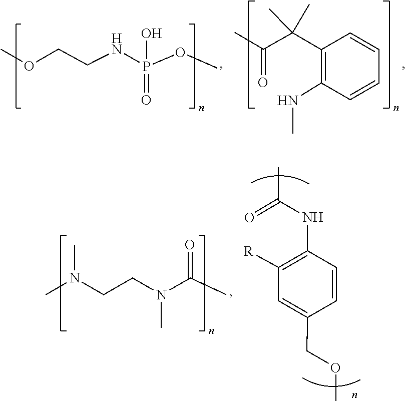

wherein R is H or alkyl, and

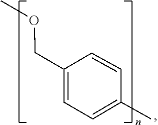

wherein n is 1 or more.

In some examples, the trigger includes an azide. In some examples, the initiator reduces the azide to a primary amine that degrades the elongated body. In some examples, the initiator includes a phosphine. In some examples, the azide is located at the second end of the elongated body. In some examples, the azide is located along the elongated body, between the first end and the second end.

In some examples, the trigger includes a secondary amine. In some examples, the initiator converts the secondary amine to a primary amine that degrades the elongated body. In some examples, the secondary amine includes:

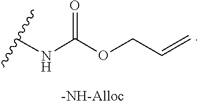 [SMILES: *NC(=O)OCC=C ; "-NH-Alloc"]

In some examples, the initiator includes a Pd⁰-phosphine complex. In some examples, the secondary amine includes:

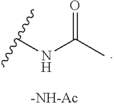 [SMILES: *NC(C)=O]

In some examples, the initiator includes an acylase enzyme. In some examples, the secondary amine includes:

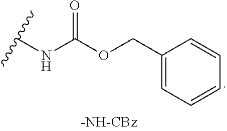 [SMILES: *NC(=O)OCc1ccccc1]

In some examples, the initiator includes palladium bound to activated carbon (Pd—C) and H₂.

In some examples, the secondary amine includes:

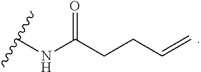 [SMILES: *NC(=O)CCC=C]

In some examples, the initiator includes N,N′-dibromodimethylhydantoin (DBDMH).

In some examples, the trigger includes —NO₂. In some examples, the initiator converts the —NO₂ to a primary amine that degrades the elongated body. In some examples, the initiator includes a palladium catalyst or a nitroreductase enzyme. In some examples, the —NO₂ is located at the second end of the elongated body.

In some examples, the trigger includes:

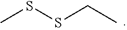

In some examples, the initiator converts the trigger to a thiol that degrades the elongated body. In some examples, the initiator includes a phosphine. In some examples, the trigger is located along the elongated body.

In some examples, the trigger includes allyloxymethoxy (AOM):

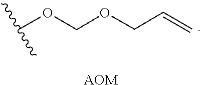

In some examples, the initiator converts the AOM to an alcohol that degrades the elongated body. In some examples, the initiator includes a Pd⁰-phosphine complex.

In some examples, the trigger includes:

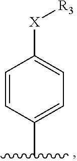

where X is O or NH, and wherein R₃ is H or a protecting group if X is O, and wherein R₃ is H or alkyl if X is NH. In some examples, the initiator converts the trigger to:

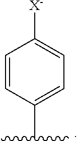

In some examples, the trigger is located at the second end of the elongated body.

In some examples, the second end includes a target, the method further including binding the target by a protein including the initiator. In some examples, the target includes biotin, and the protein includes streptavidin. In some examples, the initiator includes a phosphine. In some examples, the trigger includes an azide. In some examples, the initiator converts the azide to a primary amine that degrades the elongated body. In some examples, the trigger includes a disulfide. In some examples, the initiator converts the disulfide to a thiol that degrades the elongated body.

In some examples, 3′-blocking group is at least about 2 nm long. In some examples, the nanopore includes a biological nanopore. In some examples, the nanopore includes a solid-state nanopore.

Some examples herein provide a method of synthesizing a first polynucleotide using a nanopore. The nanopore may include a first side, a second side, and an aperture extending through the first and second sides. The method may include (a) disposing a second polynucleotide through the aperture of a nanopore such that a 3′ end of the second polynucleotide is on the first side of the nanopore, and a 5′ end of the second polynucleotide is on the second side of the nanopore. The method may include (b) forming a duplex with the second polynucleotide on the first side of the nanopore, the duplex including a 3′ end. The method may include (c) extending the duplex on the first side of the nanopore by adding a nucleotide to the 3′ end of the duplex, the nucleotide being coupled to a 3′-blocking group including a trigger. The method may include (d) selectively activating the trigger. The method may include (e) using the activated trigger to remove 3′-blocking group from the nucleotide. The method may include (f) repeating operations (c) through (e) to further extend the duplex by a plurality of additional nucleotides.

In some examples, the method further includes moving the trigger through the aperture to a second side of the nanopore, while retaining the nucleotide on the first side of the nanopore. In some examples, the trigger is activated using an initiator that is located on the second side of the nanopore and substantially not located on the first side of the nanopore.

Some examples herein provide a modified nucleotide, having the structure:

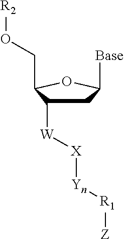

wherein W includes O or NH₂, X includes an optional spacer, Y includes a monomer, n is at least one, Z includes an optional extension, R₁ includes a trigger, and R₂ includes a phosphate or polyphosphate group. The trigger may be activatable by an initiator so as to degrade Yₙ and X and replace X (if included) or Y with H at 3′ position of the modified nucleotide.

In some examples, Yₙ is selected from the group consisting of:

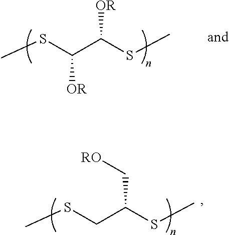

where n is at least two, and R is H, SO³⁻, or PO₃²⁻. In some examples, R₁ is selected from the group consisting of

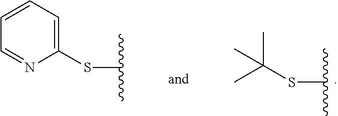

In some examples, Yₙ is selected from the group consisting of:

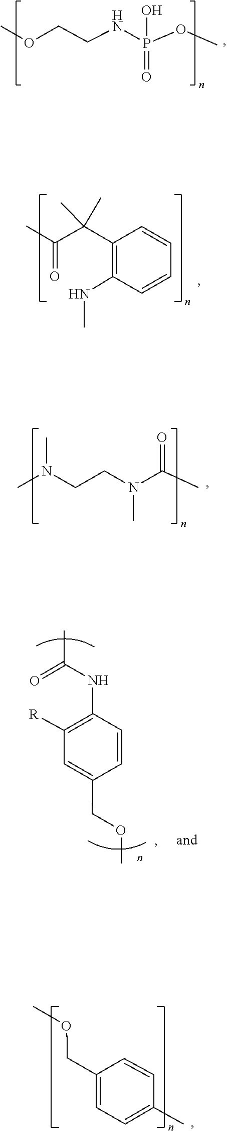

where R is H or alkyl.

In some examples, R₁ is selected from the group consisting of: an azide, a secondary amine, —NO₂,

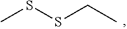

allyloxymethoxy (AOM)

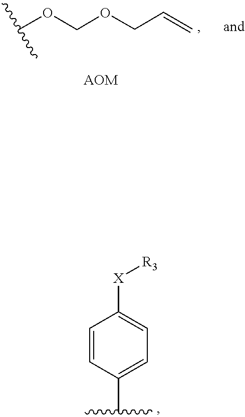

where X is O or NH, and wherein R₃ is H or a protecting group if X is O, and wherein R₃ is H or alkyl if X is NH.

In some examples, the secondary amine is selected from the group consisting of:

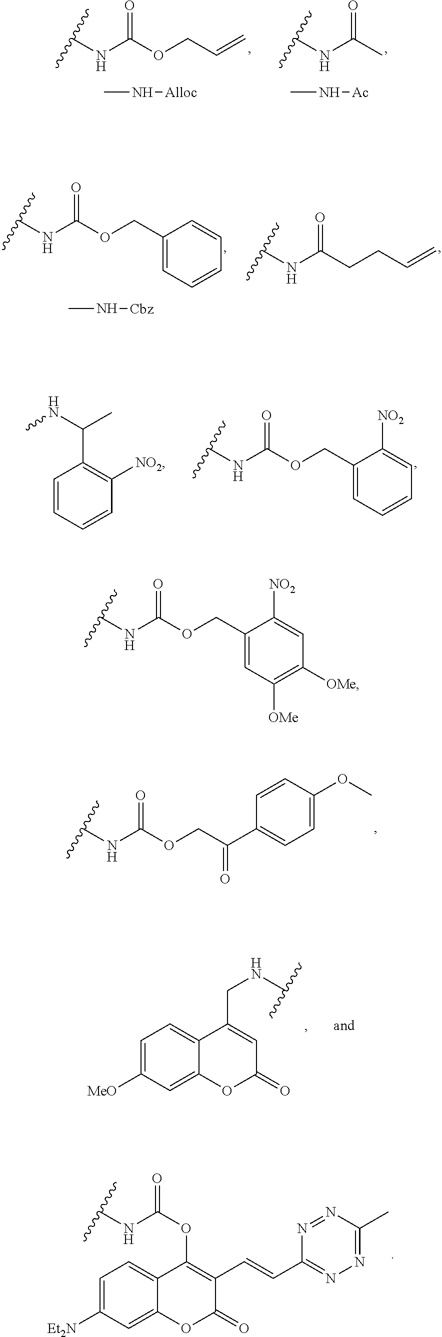

In some examples, Z includes a target. Some examples further include a protein binding the target. In some examples, the target includes biotin, and the protein includes streptavidin. In some examples, the protein is coupled to a phosphine.

In some examples, X (if included), Yₙ, Z (if included), and R₁, together, are at least about 2 nm long.

Some examples herein provide a composition that includes any of the foregoing modified nucleotides and a nanopore having a first side and a second side, wherein the nucleotide is located on the first side of the nanopore and at least R₁ is located on the second side of the nanopore.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIGS. 1A-1D schematically illustrate example compositions and operations for deblocking 3′-blocked nucleotides.

FIGS. 2A-2C schematically illustrate additional compositions and operations for deblocking 3′-blocked nucleotides.

FIGS. 3A-3C schematically illustrate additional compositions and operations for deblocking 3′-blocked nucleotides.

FIGS. 4A-4D schematically illustrate example initiator structures for use in deblocking 3′-blocked nucleotides.

FIGS. 5A-5B schematically illustrate example initiator structures for use in deblocking 3′-blocked nucleotides.

FIG. 6 illustrates a flow of operations in an example method for deblocking 3′-blocked nucleotides.

FIG. 7 illustrates a flow of operations in an example method for synthesizing a polynucleotide using 3′-blocked nucleotides.

### DETAILED DESCRIPTION

3′-blocked nucleotides, methods of deblocking the same, and methods of synthesizing polynucleotides using the same are provided herein.

More specifically, the present 3′-blocked nucleotides may be selectively deblocked after being incorporated into a complementary strand, without necessarily requiring a separate fluidic cycle to introduce a deblocking agent. Instead, the base of the present 3′-blocked nucleotide may be located in the aperture of a nanopore on a first side of the nanopore, and 3′-blocking group selectively may be contacted by an initiator. In some examples, the initiator is located substantially on a second, opposite side of the nanopore. In other examples, the initiator is located within the aperture of the nanopore. The initiator causes 3′-blocking group to degrade, thus replacing 3′-blocking group with a hydroxyl group (—OH) or amino group (—NH₂). Another 3′-blocked nucleotide may be added to the deblocked nucleotide, e.g., by a polymerase incorporating that nucleotide into a growing polynucleotide, and 3′-blocking group of that nucleotide then may be degraded in a similar manner. Such operations may be repeated any suitable number of times to grow the complementary strand. Because the initiator selectively deblocks the nucleotide which is in the aperture of the nanopore, the initiator may not deblock any 3′-blocked nucleotides that are in solution on the first side of the nanopore and have not yet been incorporated into the growing polynucleotide.

The present disclosure describes many different examples of monomers that may be used in 3′-blocking group. In some examples, 3′-blocking group is sufficiently long to partially extend into the second side of the nanopore where the initiator is substantially located, and that degrade (e.g., cyclize, self-immolate or perform a cascade of cyclizations) once activated. In other examples, 3′ blocking group substantially is retained within the aperture of the nanopore. The present disclosure also describes many examples of trigger groups that can be activated, using an initiator, to generate moieties such as primary amines, thiols, or alcohols that can initiate the degradation. Still further examples of monomers and triggers readily may be envisioned, and are encompassed within the present disclosure. Additionally, it will be appreciated that although the present 3′-blocked nucleotides may be used together with a nanopore, such nucleotides need not necessarily be used with a nanopore and indeed may be used in any suitable application or context.

First, some terms used herein will be briefly explained. Then, some example 3′-blocked nucleotides, and methods of deblocking the same, will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or system, the term “comprising” means that the compound, composition, or system includes at least the recited features or components, but may also include additional features or components.

As used herein, the singular forms “a”, “an” and “the” include plural referents unless the content clearly dictates otherwise.

The terms “substantially,” “approximately,” and “about” used throughout this specification are used to describe and account for small fluctuations, such as due to variations in processing. For example, they may refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar, backbone, and/or phosphate moiety compared to naturally occurring nucleotides. Nucleotide analogues also may be referred to as “modified nucleic acids.” Example modified nucleobases include inosine, xathanine, hypoxathanine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates. Nucleotide analogues also include locked nucleic acids (LNA), peptide nucleic acids (PNA), and 5-hydroxylbutynl-2′-deoxyuridine (“super T”).

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof such as locked nucleic acids (LNA) and peptide nucleic acids (PNA). A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA, LNA, or PNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primer and a single stranded target polynucleotide, and can sequentially add nucleotides to the growing primer to form a “complementary copy” polynucleotide having a sequence that is complementary to that of the target polynucleotide. DNA polymerases may bind to the target polynucleotide and then move down the target polynucleotide sequentially adding nucleotides to the free hydroxyl group at 3′ end of a growing polynucleotide strand. DNA polymerases may synthesize complementary DNA molecules from DNA templates. RNA polymerases may synthesize RNA molecules from DNA templates (transcription). Other RNA polymerases, such as reverse transcriptases, may synthesize cDNA molecules from RNA templates. Still other RNA polymerases may synthesize RNA molecules from RNA templates, such as RdRP. Polymerases may use a short RNA or DNA strand (primer), to begin strand growth. Some polymerases may displace the strand upstream of the site where they are adding bases to a chain. Such polymerases may be said to be strand displacing, meaning they have an activity that removes a complementary strand from a template strand being read by the polymerase.

Example DNA polymerases include Bst DNA polymerase, 9° Nm DNA polymerase, Phi29 DNA polymerase, DNA polymerase I (E. coli), DNA polymerase I (Large), (Klenow) fragment, Klenow fragment (3′-5′ exo-), T4 DNA polymerase, T7 DNA polymerase, Deep VentR™ (exo-) DNA polymerase, Deep VentR™ DNA polymerase, DyNAzyme™ EXT DNA, DyNAzyme™ II Hot Start DNA Polymerase, Phusion™ High-Fidelity DNA Polymerase, Therminator™ DNA Polymerase, Therminator™ II DNA Polymerase, VentR® DNA Polymerase, VentR® (exo-) DNA Polymerase, RepliPHI™ Phi29 DNA Polymerase, rBst DNA Polymerase, rBst DNA Polymerase (Large), Fragment (IsoTherm™ DNA Polymerase), MasterAmp™ AmpliTherm™, DNA Polymerase, Taq DNA polymerase, Tth DNA polymerase, Tfl DNA polymerase, Tgo DNA polymerase, SP6 DNA polymerase, Tbr DNA polymerase, DNA polymerase Beta, ThermoPhi DNA polymerase, and Isopol™ SD+ polymerase. In specific, nonlimiting examples, the polymerase is selected from a group consisting of Bst, Bsu, and Phi29. Some polymerases have an activity that degrades the strand behind them (3′ exonuclease activity). Some useful polymerases have been modified, either by mutation or otherwise, to reduce or eliminate 3′ and/or 5′ exonuclease activity.

Example RNA polymerases include RdRps (RNA dependent, RNA polymerases) that catalyze the synthesis of the RNA strand complementary to a given RNA template. Example RdRps include polioviral 3Dpol, vesicular stomatitis virus L, and hepatitis C virus NS5B protein. Example RNA Reverse Transcriptases. A non-limiting example list to include are reverse transcriptases derived from Avian Myelomatosis Virus (AMV), Murine Moloney Leukemia Virus (MMLV) and/or the Human Immunodeficiency Virus (HIV), telomerase reverse transcriptases such as (hTERT), SuperScript™ III, SuperScript™ IV Reverse Transcriptase, ProtoScript® II Reverse Transcriptase.

As used herein, the term “primer” is defined as a polynucleotide to which nucleotides may be added via a free 3′ XHn group, where X is any nucleophilic atom not limited to O, S and N, and wherein n is any integer number that is compatible with X (e.g., n may be 1, 2, or 3). A primer may include a 3′ block inhibiting polymerization until the block is removed. A primer may include a modification at the 5′ terminus to allow a coupling reaction or to couple the primer to another moiety. A primer may include one or more moieties, such as 8-oxo-G, which may be cleaved under suitable conditions, such as UV light, chemistry, enzyme, or the like. The primer length may be any suitable number of bases long and may include any suitable combination of natural and non-natural nucleotides. A target polynucleotide may include an “amplification adapter” or, more simply, an “adapter,” that hybridizes to (has a sequence that is complementary to) a primer, and may be amplified so as to generate a complementary copy polynucleotide by adding nucleotides to the free 3′ OH group of the primer.

As used herein, the term “plurality” is intended to mean a population of two or more different members. Pluralities may range in size from small, medium, large, to very large. The size of small plurality may range, for example, from a few members to tens of members. Medium sized pluralities may range, for example, from tens of members to about 100 members or hundreds of members. Large pluralities may range, for example, from about hundreds of members to about 1000 members, to thousands of members and up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above example ranges. Example polynucleotide pluralities include, for example, populations of about 1×10⁵ or more, 5×10⁵ or more, or 1×10⁶ or more different polynucleotides. Accordingly, the definition of the term is intended to include all integer values greater than two. An upper limit of a plurality may be set, for example, by the theoretical diversity of polynucleotide sequences in a sample.

As used herein, the term “double-stranded,” when used in reference to a polynucleotide, is intended to mean that all or substantially all of the nucleotides in the polynucleotide are hydrogen bonded to respective nucleotides in a complementary polynucleotide. A double-stranded polynucleotide also may be referred to as a “duplex.”

As used herein, the term “single-stranded,” when used in reference to a polynucleotide, means that essentially none of the nucleotides in the polynucleotide are hydrogen bonded to a respective nucleotide in a complementary polynucleotide.

As used herein, the term “target polynucleotide” is intended to mean a polynucleotide that is the object of an analysis or action, and may also be referred to using terms such as “library polynucleotide,” “template polynucleotide,” or “library template.” The analysis or action includes subjecting the polynucleotide to amplification, sequencing and/or other procedure. A target polynucleotide may include nucleotide sequences additional to a target sequence to be analyzed. For example, a target polynucleotide may include one or more adapters, including an amplification adapter that functions as a primer binding site, that flank(s) a target polynucleotide sequence that is to be analyzed. In particular examples, target polynucleotides may have different sequences than one another but may have first and second adapters that are the same as one another. The two adapters that may flank a particular target polynucleotide sequence may have the same sequence as one another, or complementary sequences to one another, or the two adapters may have different sequences. Thus, species in a plurality of target polynucleotides may include regions of known sequence that flank regions of unknown sequence that are to be evaluated by, for example, sequencing (e.g., SBS). In some examples, target polynucleotides carry an amplification adapter at a single end, and such adapter may be located at either 3′ end or 5′ end the target polynucleotide. Target polynucleotides may be used without any adapter, in which case a primer binding sequence may come directly from a sequence found in the target polynucleotide.

The terms “polynucleotide” and “oligonucleotide” are used interchangeably herein. The different terms are not intended to denote any particular difference in size, sequence, or other property unless specifically indicated otherwise. For clarity of description, the terms may be used to distinguish one species of polynucleotide from another when describing a particular method or composition that includes several polynucleotide species.

As used herein, the term “substrate” refers to a material used as a support for compositions described herein. Example substrate materials may include glass, silica, plastic, quartz, metal, metal oxide, organo-silicate (e.g., polyhedral organic silsesquioxanes (POSS)), polyacrylates, tantalum oxide, complementary metal oxide semiconductor (CMOS), or combinations thereof. An example of POSS can be that described in Kehagias et al., Microelectronic Engineering 86 (2009), pp. 776-778, which is incorporated by reference in its entirety. In some examples, substrates used in the present application include silica-based substrates, such as glass, fused silica, or other silica-containing material. In some examples, silica-based substrates can include silicon, silicon dioxide, silicon nitride, or silicone hydride. In some examples, substrates used in the present application include plastic materials or components such as polyethylene, polystyrene, poly(vinyl chloride), polypropylene, nylons, polyesters, polycarbonates, and poly(methyl methacrylate). Example plastics materials include poly(methyl methacrylate), polystyrene, and cyclic olefin polymer substrates. In some examples, the substrate is or includes a silica-based material or plastic material or a combination thereof. In particular examples, the substrate has at least one surface including glass or a silicon-based polymer. In some examples, the substrates can include a metal. In some such examples, the metal is gold. In some examples, the substrate has at least one surface including a metal oxide. In one example, the surface includes a tantalum oxide or tin oxide. Acrylamides, enones, or acrylates may also be utilized as a substrate material or component. Other substrate materials can include, but are not limited to gallium arsenide, indium phosphide, aluminum, ceramics, polyimide, quartz, resins, polymers and copolymers. In some examples, the substrate and/or the substrate surface can be, or include, quartz. In some other examples, the substrate and/or the substrate surface can be, or include, semiconductor, such as GaAs or ITO. The foregoing lists are intended to be illustrative of, but not limiting to the present application. Substrates can include a single material or a plurality of different materials. Substrates can be composites or laminates. In some examples, the substrate includes an organo-silicate material.

Substrates can be flat, round, spherical, rod-shaped, or any other suitable shape. Substrates may be rigid or flexible. In some examples, a substrate is a bead or a flow cell.

Substrates can be non-patterned, textured, or patterned on one or more surfaces of the substrate. In some examples, the substrate is patterned. Such patterns may include posts, pads, wells, ridges, channels, or other three-dimensional concave or convex structures. Patterns may be regular or irregular across the surface of the substrate. Patterns can be formed, for example, by nanoimprint lithography or by use of metal pads that form features on non-metallic surfaces, for example.

In some examples, a substrate described herein forms at least part of a flow cell or is located in or coupled to a flow cell. Flow cells may include a flow chamber that is divided into a plurality of lanes or a plurality of sectors. Example flow cells and substrates for manufacture of flow cells that can be used in methods and compositions set forth herein include, but are not limited to, those commercially available from Illumina, Inc. (San Diego, CA).

As used herein, the term “electrode” is intended to mean a solid structure that conducts electricity. Electrodes may include any suitable electrically conductive material, such as gold, palladium, or platinum, or combinations thereof. In some examples, an electrode may be disposed on a substrate. In some examples, an electrode may define a substrate.

As used herein, the term “particle” is intended to mean a solid structure that is made up of a large number of atoms (e.g., more than about 100 atoms) and has a three dimensional structure with at least one external dimension being larger than the smallest dimension of an aperture of a nanopore, e.g., about 2 nm. In some examples, a particle has a three dimensional structure with at least two external dimensions being larger than the smallest dimension of an aperture of a nanopore, e.g., about 2 nm. In some examples, a particle has a three dimensional structure with all three external dimensions being larger than the smallest dimension of an aperture of a nanopore, e.g., about 2 nm. Nonlimiting examples of particles include beads and scaffolds that are optionally permeable.

In some examples, a particle may act as a single unit with regards to its translational transport properties in a fluid. For example, translational movement of a first portion of the particle causes other portions of the particle to translationally move concurrently in the fluid. In comparison, an elongated, flexible, two-dimensional structure (such as a polymer lacking tertiary structure) may not necessarily act as a single unit with regards to its translational transport properties a fluid. For example, translational movement of a first end of such a structure may not cause translational movement of a second end of such a structure.

Some particles herein may include, or may consist of, a single molecule such as a polymer that has a tertiary structure. As used herein, a particle with “tertiary structure” is intended to mean a particle that is folded into a three-dimensional tertiary structure having internal cross-linking holding the folds in place. In comparison, a polymer that has a primary structure (e.g., a particular sequence of monomers linked together) and a secondary structure (e.g., local structure) but no internal cross-linking holding folds into place would not be considered to have a tertiary structure as the term is used herein, nor would that polymer be considered to be a particle as the term is used herein. For example, a double-stranded polynucleotide (e.g., dsDNA), a single-stranded polynucleotide (e.g., ssDNA), or a partially double-stranded (e.g., part dsDNA and part ssDNA) that has a primary structure (a particular sequence of bases in each of the strands) and a secondary structure (e.g., a double helix) but that is not folded and cross-linked into a tertiary structure is not considered to be a “particle” as the term is used herein. In comparison, a single-stranded, double-stranded, or partially double-stranded polynucleotide with a tertiary structure, or a polypeptide chain with a tertiary structure, may be considered to be a “particle” as the term is used herein.

Particles herein may include, or may consist of, a collection of discrete atoms or molecules that are attached to one another, e.g., are bonded to one another. An example of such a particle is a nanoparticle. Nanoparticles have one or more outer dimensions in the range of about 5 to about 100 nm, or two or more outer dimensions in the range of about 5 to about 100 nm, and in some examples have all outer dimensions in the range of about 5 to about 100 nm. By “outer dimension” it is meant a distance between outer surfaces of a particle in one direction. Nanoparticles may be spherical, or may be aspherical. Spherical or approximately spherical nanoparticles may have a diameter of about 5 to about 100 nm. Aspherical nanoparticles may be regularly shaped, e.g., may be elongated, or may be irregularly shaped. Aspherical nanoparticles may be referred to as having a diameter, even though they are not spherical. The diameter of an aspherical particle may refer to an average value of at least one dimension of the particle, and in some examples may refer to an average value of all dimensions of the particle. An elongated nanoparticle may have a diameter of about 5 to about 100 nm and a length greater than about 100 nm.

Particles may be electrically conductive, semiconductive, or electrically nonconductive (e.g., may be electrical insulators). Particles may include any suitable material or combination of materials. Electrically conductive particles may include, for example, gold, platinum, carbon, silver, palladium, or the like. Semiconductive particles may include one or more materials including, for example, cadmium, zinc, titanium, mercury, manganese, sulfur, selenium, tellurium, carbon, or the like. Electrically nonconductive particles may include, for example, silicon oxide, iron oxide, aluminum oxide, organic polymers, proteins, or the like. Hybrid particles may include a combination of electrically conductive, semiconductive, and/or electrically nonconductive materials.

Particles may include or may be coupled to functional groups. By “functional group” it is meant a molecular moiety that has one end bonded to the surface of the particle and has another end extending away from the surface of the molecule which may bond to another structure.

As used herein, the term “nanopore” is intended to mean a structure that includes an aperture that permits molecules to cross therethrough from a first side of the nanopore to a second side of the nanopore, in which a portion of the aperture of a nanopore has a width of 100 nm or less, e.g., 10 nm or less, or 2 nm or less. The aperture extends through the first and second sides of the nanopore. Molecules that can cross through an aperture of a nanopore can include, for example, ions or water-soluble molecules such as amino acids or nucleotides. The nanopore can be disposed within a barrier, or can be provided through a substrate. Optionally, a portion of the aperture can be narrower than one or both of the first and second sides of the nanopore, in which case that portion of the aperture can be referred to as a “constriction.” Alternatively or additionally, the aperture of a nanopore, or the constriction of a nanopore (if present), or both, can be greater than 0.1 nm, 0.5 nm, 1 nm, 10 nm or more. A nanopore can include multiple constrictions, e.g., at least two, or three, or four, or five, or more than five constrictions. nanopores include biological nanopores, solid-state nanopores, or biological and solid-state hybrid nanopores.

Biological nanopores include, for example, polypeptide nanopores and polynucleotide nanopores. A “polypeptide nanopore” is intended to mean a nanopore that is made from one or more polypeptides. The one or more polypeptides can include a monomer, a homopolymer or a heteropolymer. Structures of polypeptide nanopores include, for example, an α-helix bundle nanopore and a β-barrel nanopore as well as all others well known in the art. Example polypeptide nanopores include α-hemolysin, Mycobacterium smegmatis porin A, gramicidin A, maltoporin, OmpF, OmpC, PhoE, Tsx, F-pilus, SP1, mitochondrial porin (VDAC), Tom40, outer membrane phospholipase A, CsgG, and Neisseria autotransporter lipoprotein (NaIP). Mycobacterium smegmatis porin A (MspA) is a membrane porin produced by Mycobacteria, allowing hydrophilic molecules to enter the bacterium. MspA forms a tightly interconnected octamer and transmembrane beta-barrel that resembles a goblet and includes a central constriction. For further details regarding α-hemolysin, see U.S. Pat. No. 6,015,714, the entire contents of which are incorporated by reference herein. For further details regarding SP1, see Wang et al., Chem. Commun., 49:1741-1743 (2013), the entire contents of which are incorporated by reference herein. For further details regarding MspA, see Butler et al., “Single-molecule DNA detection with an engineered MspA protein nanopore,” Proc. Natl. Acad. Sci. 105:20647-20652 (2008) and Derrington et al., “Nanopore DNA sequencing with MspA,” Proc. Natl. Acad. Sci. USA, 107:16060-16065 (2010), the entire contents of both of which are incorporated by reference herein. Other nanopores include, for example, the MspA homolog from Norcadia farcinica, and lysenin. For further details regarding lysenin, see PCT Publication No. WO 2013/153359, the entire contents of which are incorporated by reference herein.

A “polynucleotide nanopore” is intended to mean a nanopore that is made from one or more nucleic acid polymers. A polynucleotide nanopore can include, for example, a polynucleotide origami.

A “solid-state nanopore” is intended to mean a nanopore that is made from one or more materials that are not of biological origin. A solid-state nanopore can be made of inorganic or organic materials. Solid-state nanopores include, for example, silicon nitride (SiN), silicon dioxide (SiO₂), silicon carbide (SiC), hafnium oxide (HfO₂), molybdenum disulfide (MoS₂), hexagonal boron nitride (h-BN), or graphene. A solid-state nanopore may comprise an aperture formed within a solid-state membrane, e.g., a membrane including any such material(s).

A “biological and solid-state hybrid nanopore” is intended to mean a hybrid nanopore that is made from materials of both biological and non-biological origins. Materials of biological origin are defined above and include, for example, polypeptides and polynucleotides. A biological and solid-state hybrid nanopore includes, for example, a polypeptide-solid-state hybrid nanopore and a polynucleotide-solid-state nanopore.

As used herein, a “barrier” is intended to mean a structure that normally inhibits passage of molecules from one side of the barrier to the other side of the barrier. The molecules for which passage is inhibited can include, for example, ions or water soluble molecules such as nucleotides and amino acids. However, if a nanopore is disposed within a barrier, then the aperture of the nanopore may permit passage of molecules from one side of the barrier to the other side of the barrier. As one specific example, if a nanopore is disposed within a barrier, the aperture of the nanopore may permit passage of molecules from one side of the barrier to the other side of the barrier. Barriers include membranes of biological origin, such as lipid bilayers, and non-biological barriers such as solid-state membranes or substrates.

As used herein, “of biological origin” refers to material derived from or isolated from a biological environment such as an organism or cell, or a synthetically manufactured version of a biologically available structure.

As used herein, “solid-state” refers to material that is not of biological origin.

As used herein, a “blocking moiety” is intended to mean a moiety that inhibits a polymerase from adding another nucleotide to an end of a duplex until that moiety is altered or removed. A “blocking group” is a nonlimiting example of a blocking moiety, and is intended to mean a chemical group. In some examples, a nucleotide may be coupled to a blocking group. Removal of a blocking group from a nucleotide may be referred to as “deblocking” that nucleotide. In examples in which 3′ position of a nucleotide is coupled to a blocking group, that blocking group may be referred to as a “3′-blocking group.” A 3′-blocking group may inhibit a polymerase from coupling another nucleotide to that nucleotide until that moiety is removed and replaced with a hydroxyl (—OH) or amino (—NH₂) group. In nonlimiting examples, a 3′-blocking group may include a first end coupled to the nucleotide, a second end, and an elongated body therebetween.

As used herein, an “elongated body” is intended to mean a portion of a member that extends between a first end and a second end. An elongated body can be formed of any suitable material of biological origin or nonbiological origin, or a combination thereof. In some examples the elongated body may include a monomer, and in some examples may include a plurality of monomers.

As used herein, the term “trigger” is intended to mean a chemical entity that is substantially unreactive until it reacts with an “initiator” under a specified set of conditions, after which the trigger is referred to as an “activated trigger.” An “initiator” may include a biological entity (such as an enzyme) suitable to activate the trigger, or a chemical entity suitable to activate the trigger.

To “selectively” activate a trigger is intended to mean to activate that trigger and substantially not activate another trigger. For example, an initiator that selectively activates the trigger of a 3′-blocking group of a nucleotide at 3′ end of a duplex may activate that trigger, and substantially may not activate the triggers of nucleotides in solution.

As used herein, the term “degrading” is intended to mean separating into constituent parts or into simpler compounds. Such “degrading” may be initiated using a trigger that is activated by an initiator. A nonlimiting example of “degrading” is cyclization of a monomer. Another nonlimiting example of “degrading” is cascading cyclizations of a plurality of monomers that are coupled to one another. By “cascading cyclizations” it is meant that cyclization of a given one of the monomers initiates cyclization of another one of the monomers. Such initiation of cyclization of a given one of the monomers responsive to cyclization of another one of the monomers may continue until all of the repeating units are cyclized. Another nonlimiting example of “degrading” is “self-immolation” of a monomer, e.g., of a plurality of monomers that are coupled to one another. By “self-immolation” it is meant that the monomer or monomers revert(s) to it or their base unit component(s).

Illustratively, in examples in which a plurality of monomers self-immolates, the monomers of the plurality may depolymerize end-to end. For further details regarding self-immolation, see the following references, the entire contents of each of which are incorporated by reference herein: Pal et al., “Synthesis and closed-loop recycling of self-immolative poly(dithiothreitol),” Macromolecules 53 (12): 4685-4691 (2010); Bej et al., “Glutathione triggered cascade degradation of an amphiphilic poly(disulfide)-drug conjugate and targeted release,” Bioconjugate Chem. 30 (1): 101-110 (2019); and Peterson et al., “Controlled depolymerization: Stimuli-responsive self-immolative polymers,” Macromolecules 45 (18): 7317-7328 (2012).

As used herein, the term “monomer” is intended to mean a moiety that occurs at least once within an entity, such as within a 3′-blocking group. A monomer may be referred to herein as Yₙ, where Y represents the monomer and n represents the number of times (e.g., at least one) that Y occurs within the entity. Where the entity includes a plurality of monomers, the monomers may be coupled directly to one another, and as such the entity including those monomers may be considered to be a polymer. An entity may include different monomers.

As used herein, the term “spacer” is intended to mean a moiety that couples another moiety, such as a monomer of a 3′-blocking group, to 3′ position of a nucleotide.

As used herein, the term “extension” is intended to mean a moiety within an entity, such as within a 3′-blocking group, that extends beyond any monomer within that entity. The extension may form a second end of an elongated body.

### 3′-Blocked Nucleotides and Methods of Deblocking the Same

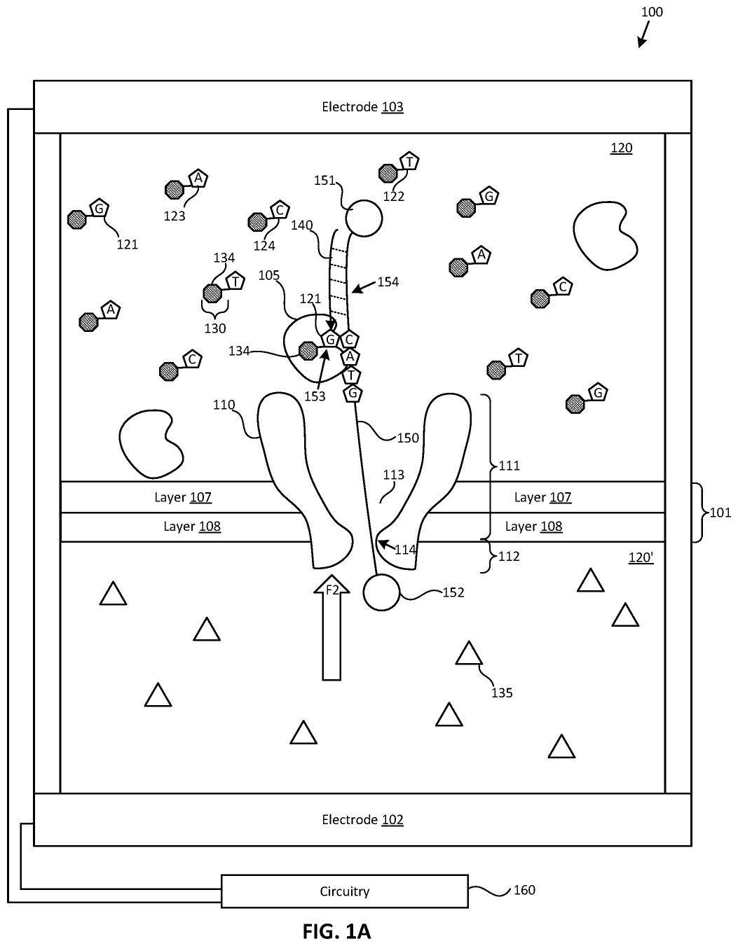

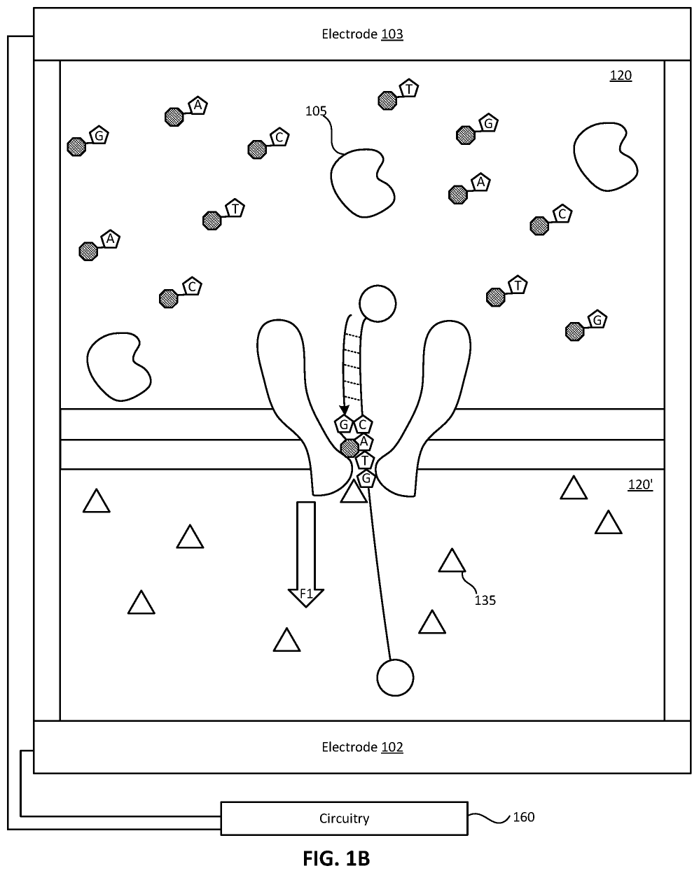

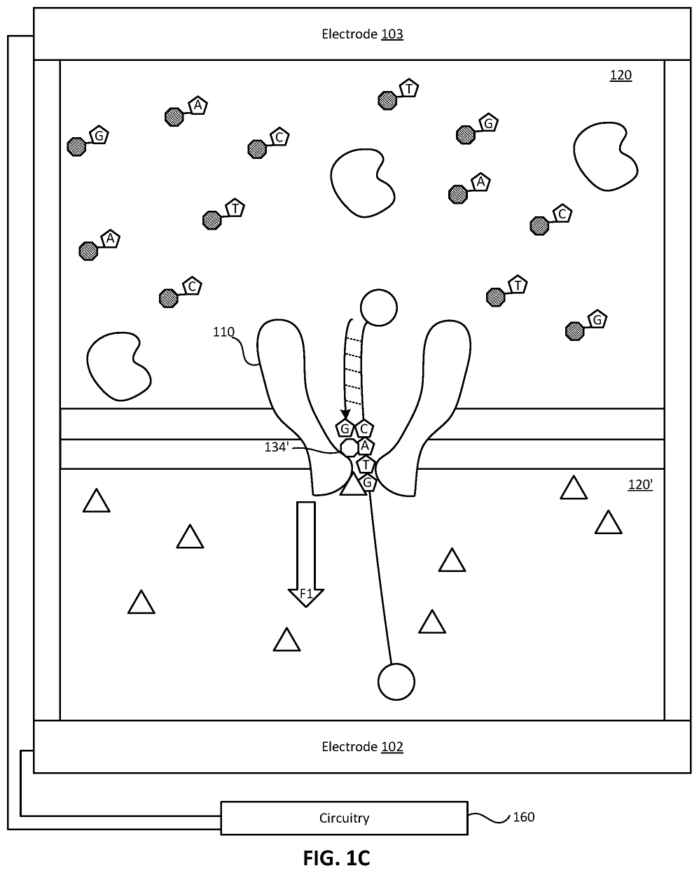

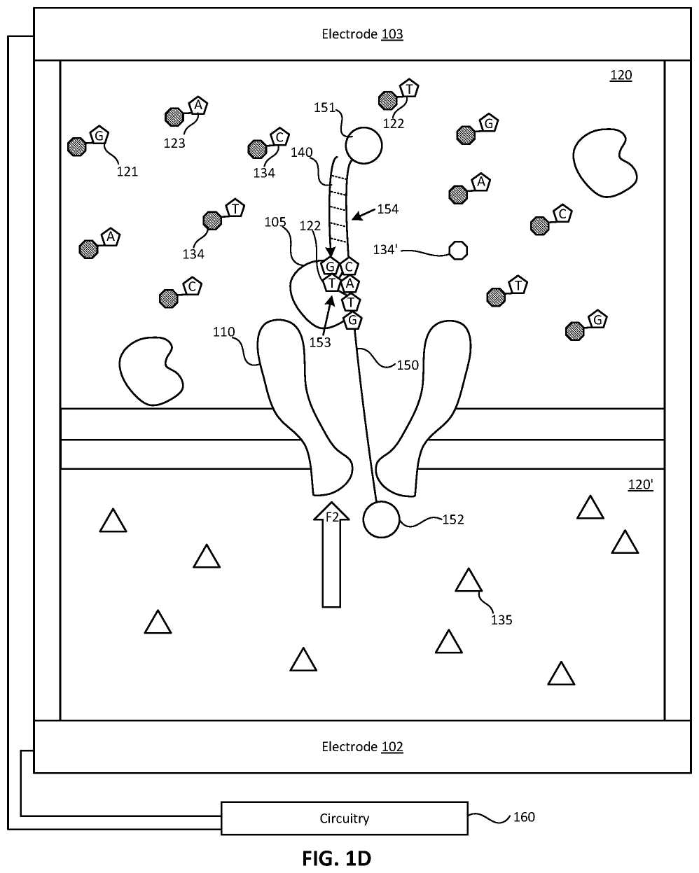

Some example 3′-blocked nucleotides and methods of deblocking the same will be described with FIGS. 1A-1D, 2A-2C, 3A-3C, 4A-4D, 5A-5B, and 6.

FIGS. 1A-1D schematically illustrate example compositions and operations for deblocking 3′-blocked nucleotides. Composition 100 illustrated in cross-section in FIG. 1A includes barrier 101; nanopore 110; fluid 120; fluid 120′; and circuitry 160 coupled to electrodes 102, 103 and configured to apply a bias voltage across the electrodes.

Barrier 101 may have any suitable structure that normally inhibits passage of molecules from one side of the barrier to the other side of the barrier, e.g., that normally inhibits contact between fluid 120 and fluid 120′. For example, as illustrated in FIG. 1A, barrier 101 may include first layer 107 and second layer 108, one or both of which inhibit the flow of molecules across that layer. Illustratively, barrier 101 may include a lipid bilayer including lipid layers 107 and 108. However, it will be appreciated that barrier 101 may include any suitable structure(s), any suitable material(s), and any suitable number of layers. For example, barrier 101 may include a solid state barrier, which may include a single layer. Nonlimiting examples of materials that may be used in barriers are provided elsewhere herein.

Nanopore 110 may be disposed within barrier 101 and may include a first side 111, a second side 112, and an aperture 113 extending through the first and second sides. As such, aperture 113 of nanopore 110 may provide a pathway for fluid 120 and/or fluid 120′ to flow through barrier 101. Nanopore 110 may include a solid-state nanopore, a biological nanopore (e.g., MspA such as illustrated in FIG. 1A), or a biological and solid state hybrid nanopore. Nonlimiting examples and properties of barriers and nanopores are described elsewhere herein, as well as in U.S. Pat. No. 9,708,655, the entire contents of which are incorporated by reference herein.

Fluid 120 may be in contact with the first side 111 of nanopore 110 and may include a plurality of each of modified nucleotides 121, 122, 123, 124, e.g., G, T, A, and C, respectively. Each of the nucleotides 121, 122, 123, 124 in fluid 120 may be coupled to a respective 3′-blocking group 130 including trigger 134. The trigger may be coupled to 3′-blocking group via one or more monomers, such as described in greater detail further below. As suggested by the darkened shading in FIG. 1A, trigger 134 is not activated at the time shown in this figure.

As provided herein, 3′-blocking group 130 may be selectively degraded using an initiator that is located on second side 112 of nanopore 101 and substantially not located on first side 111 of the nanopore 101. In some aspects, initiator is only located on second side 112 of nanopore 101. In the nonlimiting example illustrated in FIG. 1A, fluid 120′ may be in contact with second side 112 of nanopore 110 and may include initiator 135, e.g., a biological entity (such an enzyme) or a chemical entity that may react with trigger 134 in such a manner as to activate trigger 134. The activated trigger may be used to degrade 3′-blocking group 130 and provide the nucleotide with a 3′-OH or 3′-NH₂ group. Initiator 135 substantially may not be located on first side 111 of nanopore 101, e.g., substantially may not be present within fluid 120. Accordingly, initiator 135 may activate any triggers 134 located in sufficient proximity to second side 112 of nanopore 101, and substantially may not activate any triggers 135 located on first side 111 of the nanopore.

For example, at the particular time illustrated in FIG. 1A, circuitry 160 may apply a voltage bias across electrodes 102, 103 so as to apply a force F2 causing 3′ end 153 of duplex 154 between first polynucleotide 140 and second polynucleotide 150 to move out of aperture 113 of nanopore 110. Alternatively, 3′ end 153 of duplex may diffuse out of aperture 113 in the absence of an applied force. While 3′ end 153 of the duplex is sufficiently outside of the aperture 113, polymerase 105 adds nucleotide 121 (G) to 3′-end 153 of duplex 154 based on the sequence of second polynucleotide 150 using a polymerase. Accordingly, 3′-blocking group 130 coupled to nucleotide 121 becomes disposed at the 3′ end 153 of duplex 154, and inhibits the addition of any further nucleotides until removed in a manner such as now will be explained.

Circuitry 160 then may apply a voltage bias across electrodes 102, 103 so as to apply a force F1 disposing 3′-end 153 of duplex 154 within aperture 113. Nanopore 110 inhibits translocation of 3′ end 153 of duplex 154 to the second side of the nanopore while force F1 is applied. For example, at the particular time illustrated in FIG. 1B, force F1 moves duplex 154 towards the second side 112 of nanopore 110, while constriction 114 or other feature of nanopore 110 inhibits the passage of 3′ end 153 of the duplex (and thus the base of nucleotide 121) into the second side of the nanopore. Duplex 154 may be wider than constriction 114, and thus sterically hindered from passing through constriction 114. However, it should be appreciated that any suitable portion(s) of nanopore 110 may be used to inhibit duplex 154 from passing to the second side of the nanopore. Additionally, the movement of 3′ end 153 of the duplex into aperture 113 may remove polymerase 105 from duplex 154. As illustrated in FIG. 1B, application of force F1 may bring trigger 134 of 3′-blocking group 130 of nucleotide 121 into sufficient proximity to the second side 112 of the nanopore for initiator 135 to activate trigger 134 and thus initiate removal of 3′-blocking group 130 from nucleotide 121. For example, at the particular time illustrated in FIG. 1C, initiator 135 may interact with (e.g., react with) the trigger 134 of that 3′-blocking group in such a manner as to activate the trigger as suggested by the lightened shading, resulting in activated trigger 134′. Using activated trigger 134′, 3′-blocking group 130 coupled to nucleotide 121 may be degraded, e.g., such as illustrated in FIG. 1C. For example, 3′-blocking group 130 may include a monomer, or a plurality of monomers. Degrading elongated body 131 may include cyclization of a monomer, or cascading cyclizations of a plurality of monomers. Nonlimiting examples of elongated bodies including monomers that may be degraded, e.g., using cyclization, cascading cyclizations, or self-immolation, responsive to activation of a trigger by an initiator, are provided elsewhere herein.

Following removal of 3′-blocking group 130 from nucleotide 121, an additional nucleotide (e.g., nucleotide 122) from fluid 120 may be coupled to nucleotide 121. For example, as illustrated in FIG. 1D, on the first side 111 of nanopore 110, the duplex 154 between first polynucleotide 140 and second polynucleotide 150 may remain in contact with fluid 120 and with polymerase 105. The polymerase may add nucleotide 122 (coupled to a respective 3′-blocking group 130) to the 3′ end 153 of duplex 154 based on the sequence of second polynucleotide 150. The added nucleotide then may be deblocked in a manner such as described with reference to FIGS. 1B-1C, and the duplex further extended in a manner such as described with reference to FIGS. 1A and 1D so as to grow a polynucleotide having a sequence that is substantially complementary to that of polynucleotide 150.

In some examples, 3′-end of polynucleotide 150 may be located on first side 111 of nanopore 110 and may be coupled to a first locking structure 151 that is sufficiently large as not to be able to pass through aperture 113, thus retaining 3′-end of polynucleotide 150 on the first side of the nanopore. Additionally, or alternatively, 5′-end of polynucleotide 150 may be located on second side 112 of nanopore 110 and may be coupled to a second locking structure 152 that is sufficiently large as not to be able to pass through aperture 113, thus retaining 5′-end of polynucleotide 150 on the second side of the nanopore. As such, regardless of any bias voltage that circuitry 160 may apply to electrodes 102 and 103, polynucleotide 150 may remain coupled to nanopore 110. The locking structures may include any suitable structure, such as a particle or an oligonucleotide, and need not be the same as one another.

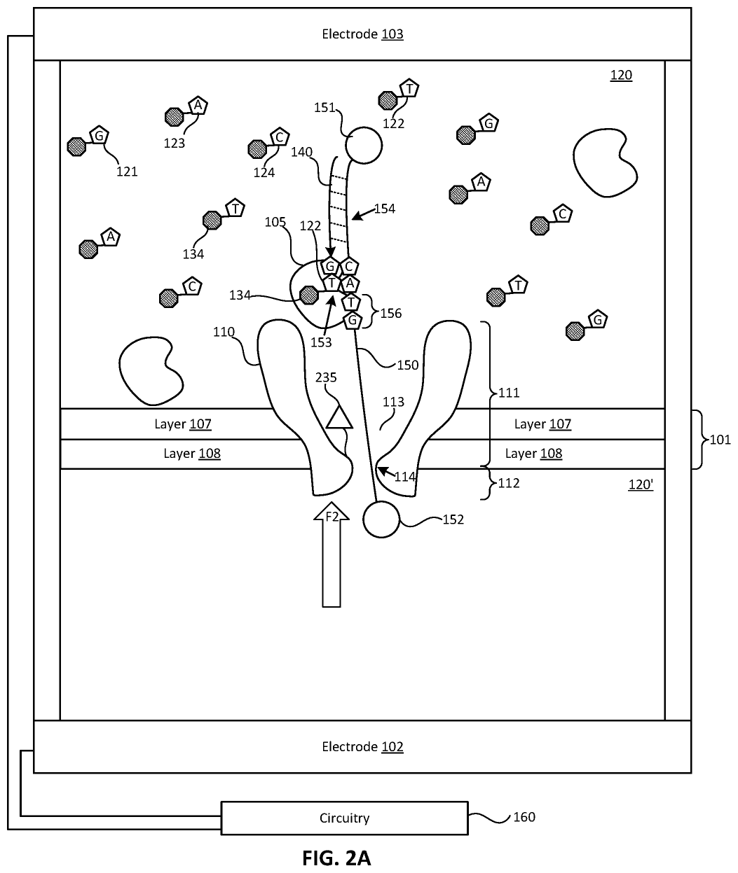

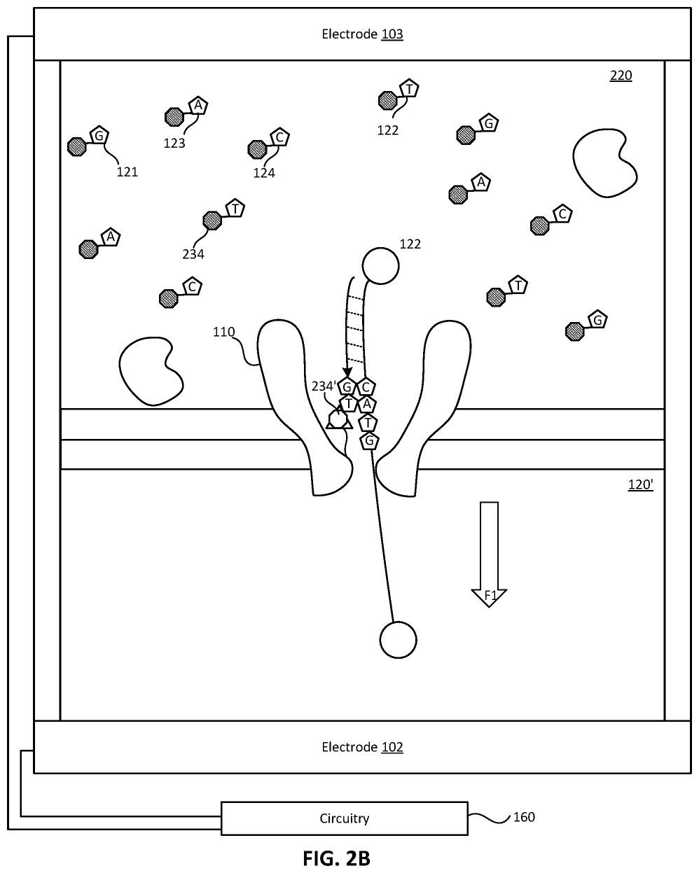

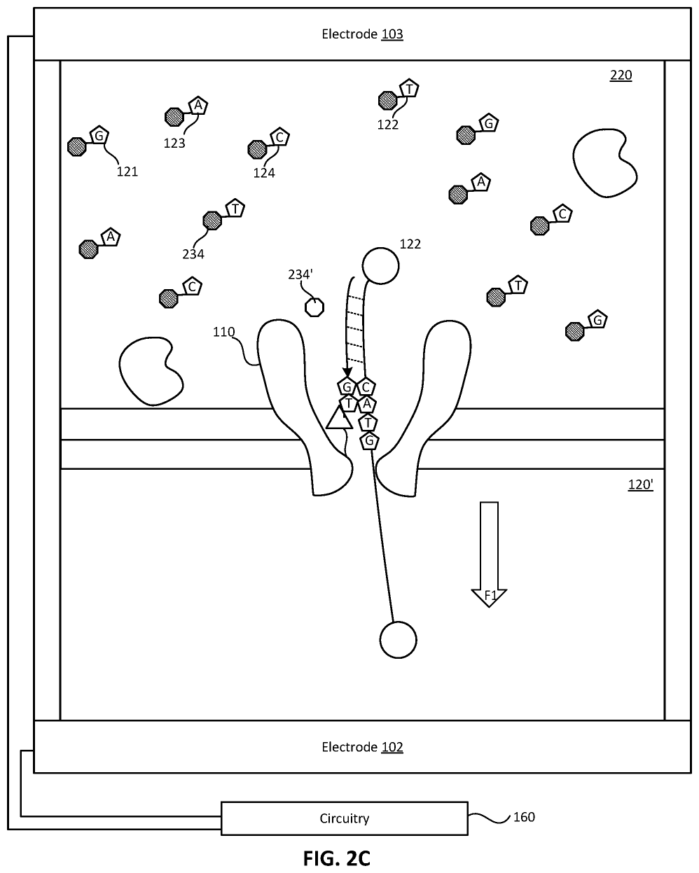

While FIGS. 1A-1D illustrate an example in which the initiator 135 is located in a fluid on the second side 111 of nanopore 110 so as to remove blocking moiety 134 while the 3′ end 153 of the duplex inside of nanopore 110, the initiator may be in any suitable location to remove the blocking moiety while 3′ end of the duplex is within the nanopore. For example, FIGS. 2A-2C additional compositions and operations for deblocking 3′ blocked nucleotides. In the example shown in FIG. 2A, initiator 135 is coupled inside the aperture 113 of nanopore 110 rather than being within fluid 120′ so that initiator 135 does not substantially interact with free nucleotides 121, 122, 123 and 124. As such, during application of second force F2 (or responsive to 3′ end 153 diffusing away from the aperture in the absence of a force) at the particular time illustrated in FIG. 2A, initiator 235 does not contact or interact with blocking moiety 134 coupled to the nucleotide at 3′ end 153 of duplex 134. Then, as illustrated in FIG. 2B, circuitry 160 applies first force F1, bringing blocking moiety 134 coupled to the nucleotide at 3′ end 153 of duplex 154 into sufficient proximity with initiator 135 to react with the blocking moiety. As illustrated in FIG. 2C, such reaction may yield modified blocking moiety 134′ which may no longer be associated with 3′ end 153 and thus may diffuse out of aperture 113.

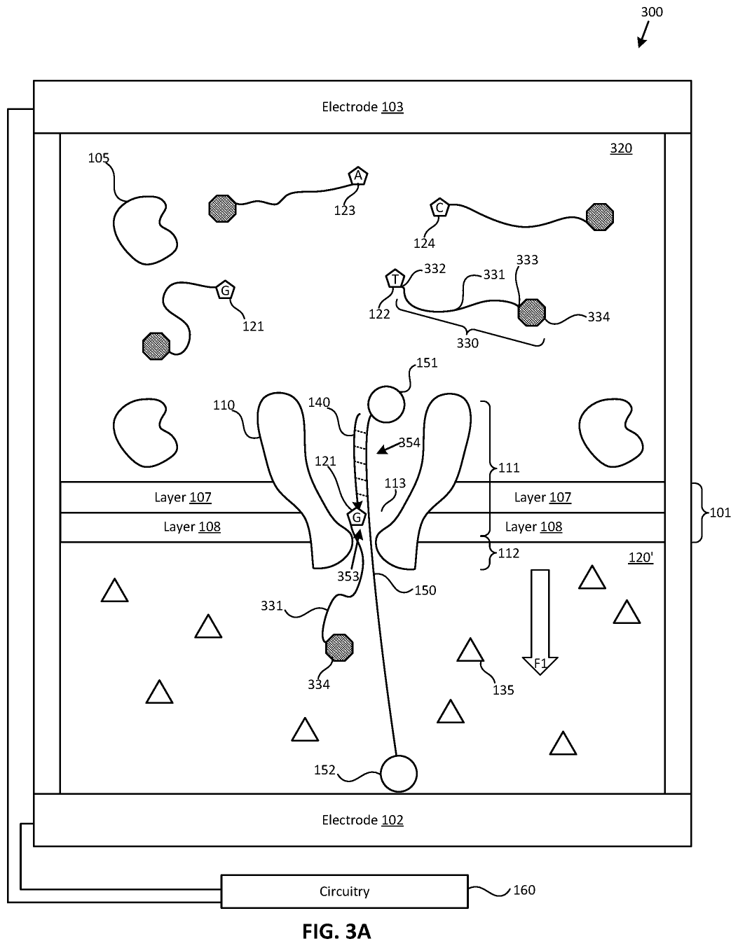

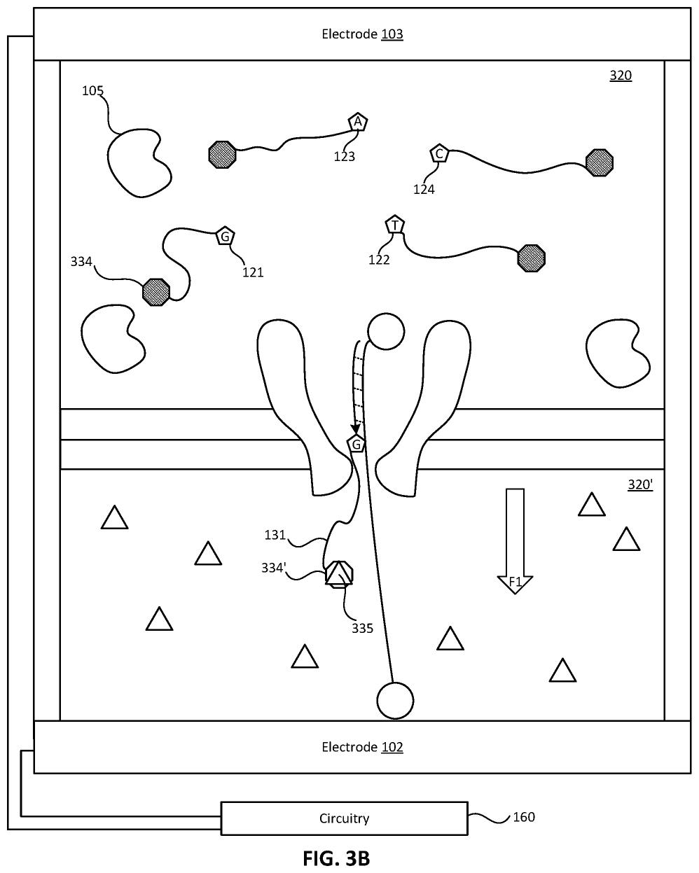

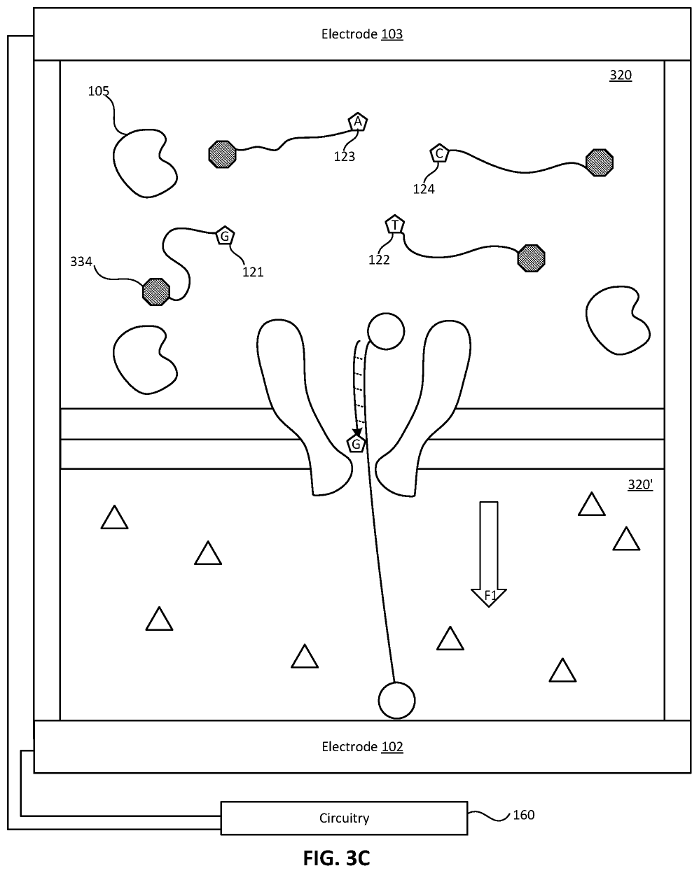

In some examples, 3′-blocking groups may be elongated such that trigger 134 becomes located on the second side of the nanopore responsive to circuitry applying force F1. For example, FIGS. 3A-3C schematically illustrate additional compositions and operations for deblocking 3′-blocked nucleotides. As illustrated in FIG. 3A, fluid 320 includes nucleotides 121, 122, 123, 124 that are coupled to respective 3′-blocking groups 330 each including an elongated body 331 including first end 332, second end 333, and trigger 334. First end 332 may be coupled to 3′-position of the respective nucleotide 121, 122, 123, 124. Trigger 334 may be coupled to any suitable portion of elongated body 331. In the nonlimiting example illustrated in FIG. 3A, trigger 334 is coupled to second end 333, while in certain other examples described elsewhere herein, trigger 334 may be coupled to elongated body 331 at a location other than second end 333. As suggested by the darkened shading in FIG. 3A, trigger 334 is not activated at the time shown in this figure.

Responsive to application of force F1, 3′-blocking group 130 coupled to nucleotide 121 may partially extend through aperture 113 of nanopore 110. For example, nucleotide 121 and the first end 332 of its respective 3′-blocking group 330 may be located on the first side 111 of nanopore 110, while trigger 334 of that 3′-blocking group may be located on second side 112 of the nanopore. Removal of 3′-blocking group 130 from nucleotide 121 may be initiated by activating trigger 334 using initiator 135. For example, at the particular time illustrated in FIG. 1B, initiator 135 may interact with (e.g., react with) the trigger 334 of that 3′-blocking group in such a manner as to activate the trigger as suggested by the lightened shading, resulting in activated trigger 334′. Using activated trigger 334′, elongated body 331 of 3′-blocking group 330 coupled to nucleotide 121 may be degraded such as illustrated in FIG. 3C. For example, 3′-blocking group 330 may include a monomer, or a plurality of monomers. Degrading elongated body 331 may include cyclization of the monomer, or cascading cyclizations of a plurality of monomers. Nonlimiting examples of elongated bodies including monomers that may be degraded, e.g., using cyclization, cascading cyclizations, or self-immolation, responsive to activation of a trigger by an initiator, are provided elsewhere herein. Another nucleotide then may be added using polymerase 150 in a manner such as described with reference to FIG. 1D, and that nucleotide then deblocked in a manner such as described with reference to FIGS. 3A-3C. Similar cycles of adding 3′-blocked nucleotides and degrading 3′-blocking groups may be repeated any suitable number of times, e.g., so as to synthesize a polynucleotide in a manner similar to that described with reference to FIGS. 1A-1D.

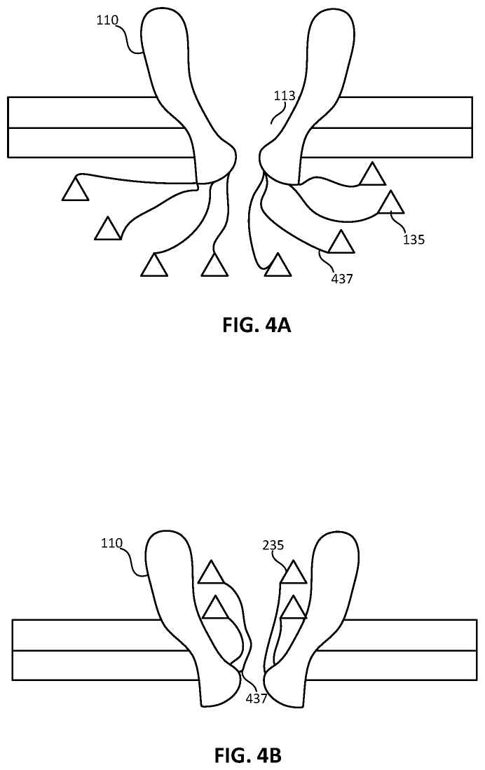

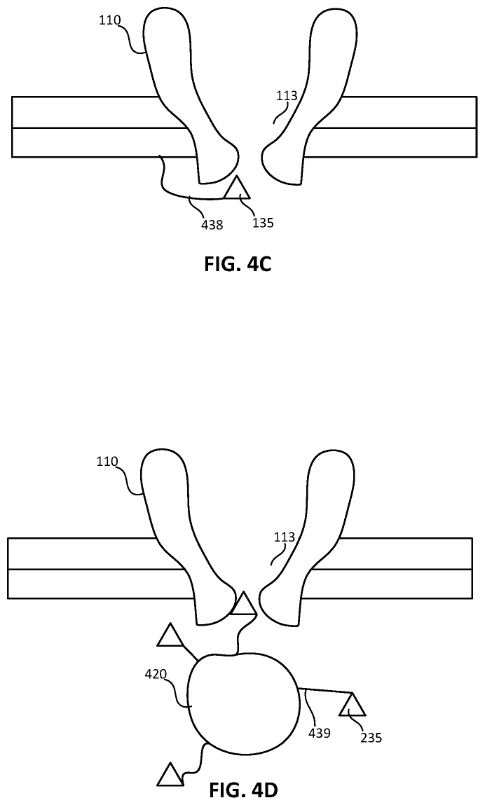

Initiator 135 may be retained substantially only on second side of nanopore 110 using any suitable structure that is larger than the narrowest portion (e.g., constriction) of nanopore 110. For example, FIGS. 4A-4D schematically illustrate example initiator structures for use in deblocking 3′-blocked nucleotides. In the example illustrated in FIG. 4A, nanopore 110 includes a plurality of residues to which respective initiator(s) 135 may be coupled via respective linkers 437. Illustratively, nanopore 110 may include MspA which is modified so as to include one or more cysteine (Cys) residues. In some examples, the thiol of such a Cys residue may be coupled to an initiator 135 via linker 437, for example using maleimide chemistry. In other examples, the sulfur of such a Cys residue may be replaced with selenium to provide a selenol (Se—H group) which acts as an initiator. Because MspA is a homo-octamer, eight such Cys residues (one for each monomer) may be provided, each of which may be coupled to a respective initiator, optionally via a linker in a manner such as illustrated in FIG. 4A. As illustrated in FIG. 4A, the initiator(s) 135 may be located on the second side of nanopore 110. Alternatively, as illustrated in FIG. 4B, the initiator(s) 135 may be located within the aperture 113 of nanopore 110 and respectively coupled to nanopore via linker 437.

In the example illustrated in FIG. 4C, one or more initiator(s) 135 may be coupled to barrier 101 via respective linkers 438. It will be appreciated that initiator(s) 135 may be directly or indirectly coupled to any suitable solid support, e.g., to substrate, to electrode 102, or other support provided in sufficient proximity to aperture 113.

In the example illustrated in FIG. 4D, particle 420 may be coupled to one or more initiators 135 via respective linkers 439. Particle 420 may be larger than the narrowest portion (e.g., constriction 114) of nanopore 110. Illustratively, particle 420 may have a diameter of at least about 2-3 nm, e.g., a diameter between about 2 and 100 nm, or a diameter between about 2 and 50 nm, or a diameter between about 2 and 20 nm, or a diameter between about 2 and 10 nm, or a diameter between about 5 and 10 nm. The particles may be monodisperse, but need not necessarily be monodisperse, so long as substantially all of the particles (e.g., more than about 70%, more than about 80%, more than about 90%, more than about 95%, or more than about 99%) are larger than the narrowest portion of nanopore 110 so as to inhibit contact between initiator 135 and nucleotides on the first side of the nanopore. Linkers 439 may be sufficiently long that initiator(s) 135 may react with trigger(s) in a manner such as described with reference to FIGS. 1A-1D, and particle 420 may be sufficiently large as to inhibit uncontrolled reactions between initiators 135 and 3′-blocking groups of nucleotides in fluid 120.

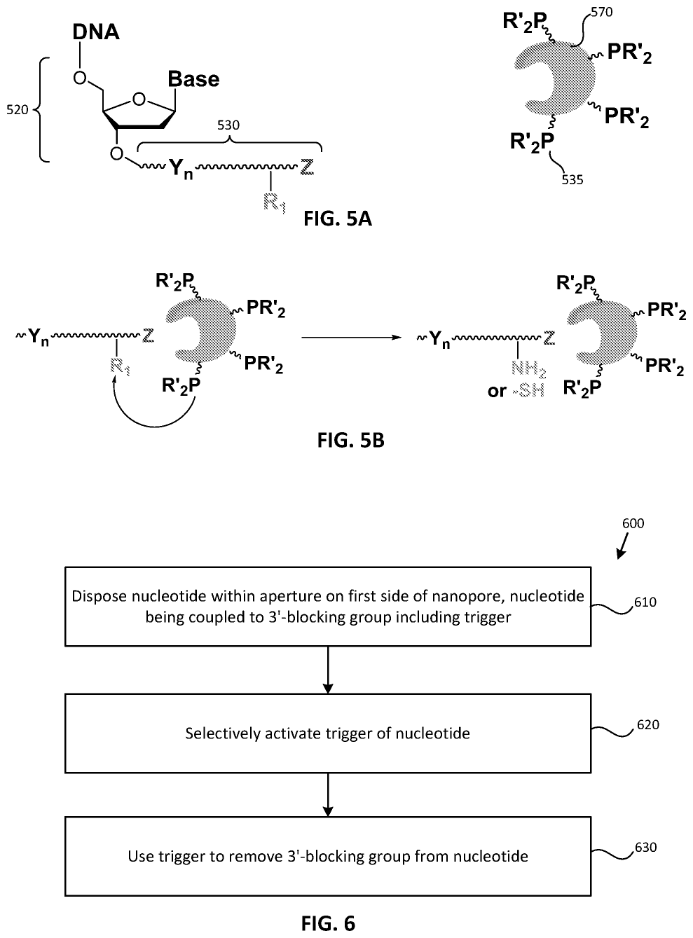

In some examples, particle 420 may include a protein within fluid 120′ in a manner such as described in greater detail below with reference to FIGS. 5A-5B. The protein optionally may be configured to bind a target which is coupled to 3′-blocking group 130. However, it should be understood that particle 220 may include any suitable material, such as a polymer, an inorganic material, or a hybrid organic-inorganic material. Some nonlimiting examples of suitable particles are provided in Table 1, below. Other example materials that may be included within particles are described elsewhere herein.

TABLE 1

| | | NP | | |
|---|---|---|---|---|
| | Synthesis of | Size | Model reaction(s) for | Efficiency |
| Type | NPs | (nm) | assessment Notes | (TOF, h⁻¹) |
| Name: Pd@PVP | Scalable | ~5 | Tsuji-Trost allylation in | >10⁴ |
| Pd NPs stabilized | synthesis using | | water. No leaching of | |
| by PVP | polyol reduction | | palladium from | |
| | procedure. | | Heterogeneous mechanism. | |
| | Na₂PdCl₄ was | | | |
| | reduced in the | | | |
| | presence of a | | | |
| | PVP stabilizer | | | |
| | with ethylene | | | |
| | glycol in a | | | |
| | microwave oven | | | |
| Name: | Sacrificial | 12 | Suzuki C-C cross- | 700-1000 |
| Pd@MCMCalixox | | | couplings | ~0.5-18 × 10³ |
| Mesoporous silica | | | Catalyzed Reduction of Ar- | ~1-5 × 10³ |
| covalently | | | NO₂ | |
| functionalized | oxidation | | Reduction | |
| with tetraallyl | | | Degradation of cationic, | |
| calixarene | | | anionic, and neutral | |
| | | | organic dyes | |
| Name: PdNPs/ | Li₂PdCl₄, | 7.1 | Sonogashira Couplings | — |
| DNA. Pd NPs | NaOAc and | | | |
| immobilized on | salmon-sperm | | | |
| DNA | DNA in | | | |
| | methanol at | | | |
| | room | | | |
| | temperature with | | | |
| | stirring for 48- | | | |
| | 72 h. | | | |
| Name: Pdnp- | | ~7.6 | Suzuki-Miyaura cross | ~70-50 |
| TPEPTA(L)-GO | | | coupling | |
| Pd NPs | | | | |
| immobilized on | | | | |
| GO | | | | |

For further details regarding preparation of nanoparticles with functional groups, see the following references, the entire contents of each of which are incorporated by reference herein: Llevot et al., “Highly efficient Tsuji-Trost allylation in water catalyzed by Pd-nanoparticles,” Chem. Commun. 53:5175-5178 (2017); Narkhede et al., “Calixarene-assisted Pd nanoparticles in organic transformations: Synthesis, characterization, and catalytic applications in water for C—C coupling and for the reduction of nitroaromatics and organic dyes,” ACS Omega 4 (3): 4908-4917 (2019); Camacho et al., “DNA-supported palladium nanoparticles as a reusable catalyst for the copper- and ligand-free Sonogashira reaction,” Catal. Sci. Technol. 7:2262-2273 (2017); and Golestanzadeh et al., “Palladium decorated on a new dendritic complex with nitrogen ligation grafted to graphene oxide: Fabrication, characterization, and catalytic application,” RSC Adv. 9:27560-27573 (2019). Particles may be commercially available. For example, nanoparticles with functional groups are commercially available from Nanopartz Inc. (Loveland, Colorado), Cerion Nanomaterials (Rochester, New York), or American Elements (Los Angeles, California).

It will be appreciated that in configurations such as described with reference to FIGS. 1A-1D, 2A-2C, 3A-3C, and 4A-4D, useful features may include one or more of the following: a stable 3′-blocking group; facile deprotection of 3′-blocking group under mild conditions; facile incorporation of 3′-blocked nucleotide onto the growing strand by DNA polymerase; a stable & highly selective initiator (deprotection reagent); and/or substantial to full isolation of the initiator (deprotection reagent) to the second side 112 of the nanopore (trans-chamber) in a manner such as described with reference to FIGS. 3A-3C and 4A-4D. In one purely illustrative, nonlimiting example, a combination of such features are achieved using allyloxymethoxy (AOM) as 3′-blocking group. For example, AOM has a relatively high heat stability, which makes it useful for enabling prolonged shelf and on-instrument life. For example, the need for cold-storage or inert conditions (e.g. oxygen- or light-free) may be reduced or eliminated. This is useful because premature deprotection/deblocking of 3′-blocking group may lead to pre-phasing (i.e. uncontrolled polymerization of free nucleotides onto the growing strand) issues during the incorporation process. Additionally, AOM is relatively easily and efficiently cleavable by an appropriate Pd catalyst under mild reaction conditions; that is, no harsh or toxic substances and conditions need be involved. Additionally, AOM is well-tolerated by DNA polymerases used for SBS, therefore reducing potential issues that may arise due to poor substrate recognition and/or slow incorporation on the growing strand.

AOM and solution-based (homogeneous) Pd catalysts readily may be used to deblock nucleotides on the first side 111 of the nanopore in a manner such as described with reference to FIGS. 1A-1D. As shown below, this is believed to proceed via a Tsuji-Trost type allylation mechanism. This involves an active Pd(0) center selectively binding to the allyl functionality on the Gen2-block of the nucleotide (Step 1) before simultaneously generating a cationic allylpalladium (II) species and a corresponding cleaved hemiacetal product (Step 2). Thereafter, a nucleophile functions as a reducing agent by adding to the allyl ligand to furnish a resultant alkene product (Step 3) which then dissociates from Pd to regenerate the active species and close the catalytic cycle (Step 4). Concomitantly, the hemiacetal product is able to undergo hydrolysis to afford the desired free 3′-OH nucleotide for the next incorporation.

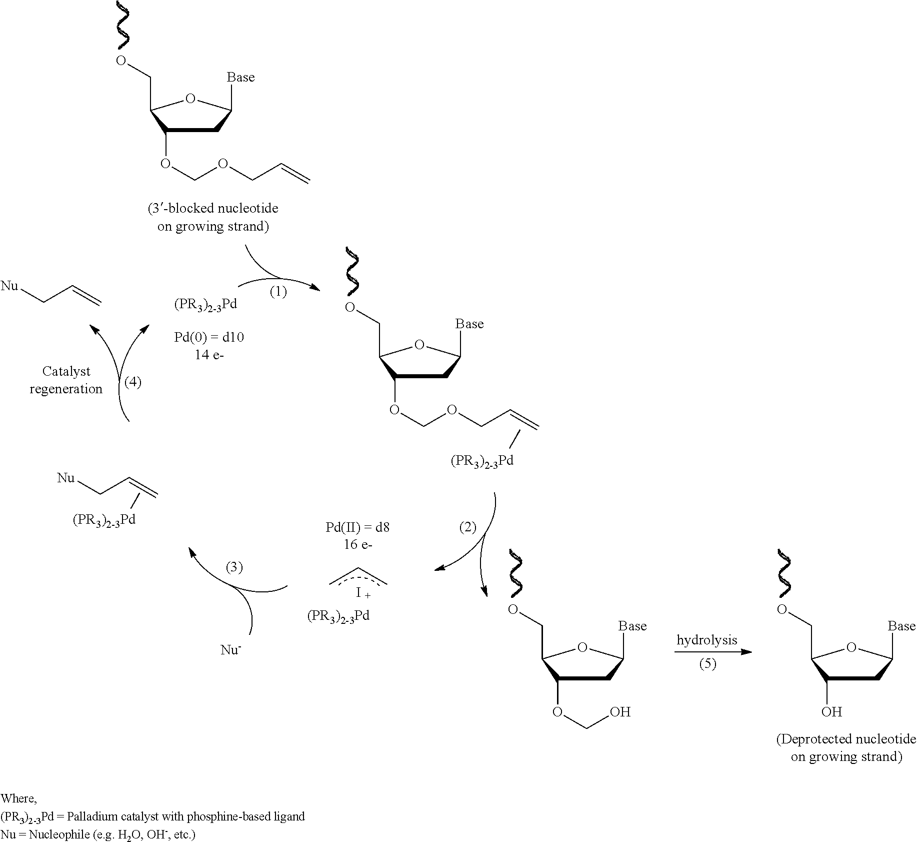

As provided herein, such AOM deblocking chemistry may be modified to use heterogeneous Pd catalysts (e.g., solid-supported Pd beads, macromolecules, or nanoparticles) instead of homogeneous, solution-based Pd catalysts. For example, heterogeneous catalysts may include a catalytic transition-metal center (e.g., Pd metal with or without phosphine-based ligands) that are immobilized and stabilized on any suitable surface of an organic or inorganic solid or polymer support matrix. Heterogeneous catalysts may provide enhanced flexibility in tuning physical and chemical properties via careful fabrication and/or modification of the solid support. See, e.g., the following references, the entire contents of each of which are incorporated by reference herein: Lamblin et al., “Recyclable heterogeneous palladium catalysts in pure water: sustainable developments in Suzuki, Heck, Sonogashira and Tsuji-Trost reactions,” Adv. Synth. Catal. 352 (1): 33-79 (2010); Yang et al., “Size- and shape-controlled palladium nanoparticles in a fluorometric Tsuji-Trost reaction,” J. Catal. 281 (1): 137-146 (2011); Llevot et al., “Highly efficient Tsuji-Trost allylation in water catalyzed by Pd-nanoparticles,” Chem. Commun. 53, 5175-5178 (2017); and Mpungose et al., “The current status of heterogeneous palladium catalysed Heck and Suzuki cross-coupling reactions,” Molecules 23 (17): 1676 (2018).

For example, solid supported catalysts may possess high reaction efficiency and repeatability due to its high surface area to volume ratio, in a manner such as described in Zapf et al., “The development of efficient catalysts for palladium-catalyzed coupling reactions of aryl halides,” Chem. Commun. 2005:431-440 (2005); and Rai et al., “Activated nanostructured bimetallic catalysts for C—C coupling reactions: recent progress,” Catal. Sci. Technol. 6:3341-3361 (2016), the entire contents of each of which are incorporated by reference herein. The solid support may be designed to exhibit improved stabilities over their homogeneous counterparts under various conditions, thus providing for greater ease of handling and operational simplicity, e.g., requiring neither heat/air/moisture-free environments nor harmful organic solvents to function. See, e.g., Llevot et al., “Highly efficient Tsuji-Trost allylation in water catalyzed by Pd-nanoparticles,” Chem. Commun. 53, 5175-5178 (2017), the entire contents of which are incorporated by reference herein. Due to their heterogeneous nature, these catalysts also may be used in relatively straightforward catalyst separation and recovery (e.g. via simple filtration) which is highly favorable from a green chemistry and recyclability perspective. See, e.g., Suzuka et al., “Reusable polymer-supported terpyridine palladium complex for Suzuki-Miyaura, Mizoroki-Heck, Sonogashira, and Tsuji-Trost reaction in water,” Polymers 3 (1): 621-639 (2011), the entire contents of which are incorporated by reference herein. The solid supported catalysts may easily be synthesized into sizes that are greater than the nanopore constriction (e.g., greater than about 1.2 nm), so that they are physically incapable of fitting through, thus inhibiting the catalyst from moving from the second side of the nanopore to the first side of nanopore through the aperture.

Via one or more structures such as described with reference to FIGS. 4A-4D (e.g., one or more residues of a nanopore, one or more particles, and/or one or more linkers to a nanopore, barrier, or particle), initiator(s) 135 are retained on the second side of the nanopore. Such structures optionally may retain initiator(s) in sufficiently close proximity to aperture 113 of nanopore 110, e.g., via linkers, as to interact with a 3′-blocking group that is within, or that at least partially extends through, aperture 113. Note that the need for fluidic cycling (the exchange of one fluid with another between certain operations) may be reduced in examples such as described with reference to FIGS. 3A-3C and 4A-4D. For example, initiator 135 may be inhibited or prevented from deblocking any of 3′-blocked nucleotides in fluid 120. Additionally, note that nucleotides in examples such as described with reference to FIGS. 1A-1D may be deblocked while 3′ end 153 of duplex 154 is disposed within aperture 113 of nanopore 110 (e.g., while circuitry 160 applies force F1 or other suitable force), even though such nucleotides may not be accessible to the solvent of fluid 120 or any elements therein (and indeed may be partially or fully inaccessible to any such elements), on the first side of the nanopore, in order to be deblocked. Nonetheless, the nucleotide at 3′ end 153 of duplex 154 may be fully or partially accessible to initiator 135 for use in deblocking the nucleotide.

It will be appreciated that fluid 120 described with reference to FIGS. 1A-1D and 2A-2C, and fluid 320 described with reference to FIGS. 3A-3E, may include any suitable combination of nucleotide analogues, ions, buffers, solvents, and the like. In some examples, fluid 120 or fluid 320 may include at least one nucleotide analogue. Each of the nucleotide analogues may include a sugar, a nucleobase, a phosphate group, and a 3′-blocking group, and such may be equivalently referred to as a nucleotide coupled to a 3′-blocking group. The nucleobase (e.g., pyrimidine or purine) and phosphate group may be directly coupled to the sugar in a standard fashion, and 3′-blocking group may be directly or indirectly coupled to the 3′ position of the sugar. For example, the nucleotide analogues may have the structure of Formula I:

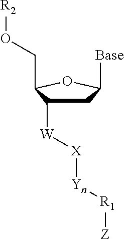

wherein W is O or NH₂, X includes an optional spacer, Y includes a monomer, n is at least one, Z includes an optional extension, R₁ includes a trigger which is optionally branched to improve trigger kinetics, and R₂ includes a phosphate or polyphosphate group which optionally is coupled to a polynucleotide strand (e.g., DNA). Together, X (if included), Yₙ, R₁, and Z (if included) may provide a 3′-blocking group that includes a first end (X if included, or Yₙ if X is not included); a second end (Z if included, or R₁ if Z is not included); and trigger R₁. The trigger may be activatable by an initiator so as to degrade Yₙ, X (if included), and Z (if included) and generate a hydroxyl or amino group at 3′ position of the modified nucleotide, e.g., in a manner such as described with reference to FIGS. 1A-1D or 3A-3E.

The 3′-blocking group, and any polymerase that may be used to add a nucleotide coupled to such a 3′-blocking group may be co-selected so as to be compatible with one another. For example, the polymerase may be able to incorporate 3′-blocked nucleotide into a growing polynucleotide at a suitable rate for the intended application or context. The 3′-blocking group and initiator may be co-selected such that the nucleotide may be deblocked at a suitable rate for the intended application or context, and such deblocking may be irreversible. The 3′-blocking group may be relatively easy to prepare and to couple to the nucleotide, and compatible with triphosphate synthesis.

Nonlimiting examples of monomers, triggers, and initiators now will be described, as well as optional spacers and optional extensions. Other examples readily may be envisioned based on the teachings provided herein.

In some examples, the initiator comprises a reducing agent. Nonlimiting examples of reducing agents include glutathione (GSH), glutathione disulfide (GSSG), seleno-glutathione (GSeH), selenoenzyme thioredoxin, NADP/NADPH, dithiothreitol (DTT) and modifications of the same, cyclodithiothreitol (cDTT), tris(hydroxypropyl)phosphine, tris(2-carboxyethyl)phosphine (TCEP), and combinations thereof. An example structure of glutathione is:

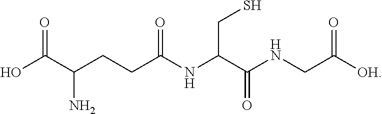 [SMILES: NC(CCC(=O)NC(CS)C(=O)NCC(=O)O)C(=O)O]

An example structure of seleno-glutathione is:

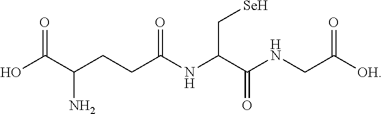

An example structure of DTT is:

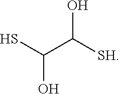

An example structure of modified DTT is:

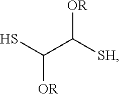

where R is any solubility-enhancing group such as SO₃ and PO₃. An example structure of cDTT is:

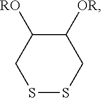

where R is any solubility-enhancing group such as H, SO₃ and PO₃.

Still other example reducing agents include other variants of reductase enzymes; thiol, selenol, or phosphine-based reducing agents; or the combinations of these or other reducing agents such as provided herein. In some examples, reducing agents may be incorporated into particles or provided as macromolecules such as polymers or biological molecules in a manner such as described with reference to FIG. 2C or 4A-4B. Still other example reducing agents are provided elsewhere herein. Based on the teachings herein, one skilled in the art readily will be able to select an appropriate reducing agent for use with a given 3′-blocking group including a given trigger. Additionally, it will be appreciated that certain reducing agents, such as DTT or GSH, may be electrochemically regenerated, for example by applying square wave pulses to electrodes 102, 103 which may include titanium. For further details, see the following references, the entire contents of each of which are incorporated by reference herein: Mao et al., “Real-time monitoring of electroreduction and labelling of disulfide-bonded peptides and proteins by mass spectrometry,” Analyst 144:6898-6904 (2019); and Shaked et al., “A combined electrochemical/enzymatic method for in situ regeneration of NADH based on cathodic reduction of cyclic disulfides,” J. Org. Chem. 46:4100-4101 (1981). The reducing agents may also or alternatively be fluidically replenished upon exhaustion from time to time.

In some examples, 3′-blocking group includes a disulfide bond. Activating the trigger may include reducing the disulfide bond, for example using one of the aforementioned reducing agents. In specific examples, 3′-blocking group includes a self-immolative tail (SIT) which may include an oligomeric structure of two or more contiguous dithiol groups (monomers Y or Formula I, where n is at least two) linked via disulfide bonds. In some examples, the SIT may be linked to the dNTP's 3′-O or 3′-NH using an intermediary mercaptoethanol (spacer X of Formula I), via (a) a carbonate or carbamate linker between the 3′-O or 3′-NH and the alcohol of the mercaptoethanol, and (b) a disulfide linker between the thiol of mercaptoethanol and a first terminal thiol of the SIT. The SIT may include any suitable dithiol groups, e.g., 1,2-dithiol groups such as DTT or dithioglycerol. In some examples, a second terminal thiol of the SIT may be coupled to a trigger (R₁ in Formula I) such as 2-mercaptopyridine,

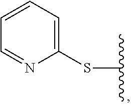 [SMILES: *Sc1ccccn1]

or may include tert-butylthiol,

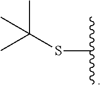 [SMILES: *SC(C)(C)C]

Responsive to an initiator reducing the disulfide bond between the thiol of the trigger and the second terminal thiol, the SIT may degrade via cascading cyclizations of the dithiol groups. Note that such cascading cyclizations may proceed significantly more quickly than the activity of polymerase 105; as such, self-immolation of the SIT is expected not to be rate-limiting in the extension of duplex 154.

It will be appreciated that the SIT based 3′-blocking group suitably may be modified so as to improve solubility, stability, and/or deblocking kinetics. For example, the dithiols optionally may be hydroxylated, sulfated, or phosphorylated so as to improve solubility and/or stability. For example, the dithiols may be DTT-based and have the structure:

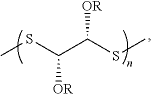

or may be dithioglycerol-based and have the structure:

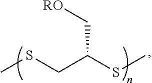

where R is H, SO³⁻, or PO₃²⁻ and n is at least one. Additionally, or alternatively, the 2-mercaptopyridine optionally may by modified to include one or more electron-withdrawing groups [e.g., —CF₃, —NO₂, —CO₂R (where R is any hydrocarbon group, halide, or the like), which may increase deblocking kinetics. Additionally, or alternatively, spacer X in Formula 1 may include one or more additional intermediary linkers, such dicarbamates, to tune reactivity and/or stability. Still other options readily may be envisioned based on the present teachings. For details regarding preparation of SITs, see Pal et al., “Synthesis and closed-loop recycling of self-immolative poly(dithiothreitol),” Macromolecules 53(12): 4685-4691 (2010) and Bej et al., “Glutathione triggered cascade degradation of an amphiphilic poly(disulfide)-drug conjugate and targeted release,” Bioconjugate Chem. 30(1): 101-110 (2019); the entire contents of each of which are incorporated by reference herein.

In some examples of a SIT based 3′-blocking group, 3′-blocking group has the structure:

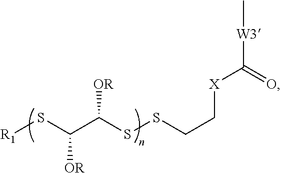

where n is at least one, W is O or NH, X is O or N, R is H, SO³⁻, or PO₃²⁻, and R₁ is selected from the group consisting of

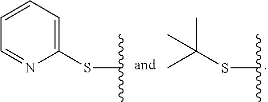

In still other examples of a SIT based 3′-blocking group, 3′-blocking group has the structure:

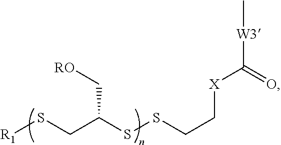

where n is at least one, W is O or NH, X is O or N, R is H, SO³⁻, or PO₃²⁻, and R₁ is selected from the group consisting of

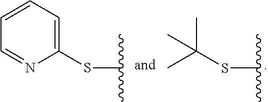

Such 3′-blocking groups may be degraded using any suitable initiator or combination of initiators, illustratively (a) a combination of GSSG and cDTT or DTT or a modification of the same, (b) GSH, (c) GSeH, or (d) a combination of selenoenzyme thioredoxin and NADP/NADPH.

In still other examples, the present 3′-blocking group may include a proximity-induced immolative tail (PIT). A PIT may include, in some examples, a single mercaptoethanol group (monomer Y in Formula I, where n equals one) directly connected to the 3′-O or 3′-NH₂ via a carbonate or carbamate linker (spacer X in Formula I). The terminal thiol of the mercaptoethanol may be coupled to a trigger (R₁ in Formula 1) such as 2-mercaptopyridine or tert-butylthiol. Responsive to an initiator reducing the disulfide bond between the thiol of the trigger and the terminal thiol, the PIT may degrade. In some examples, the initiator for a PIT includes a selenocysteine group (Sec) group coupled to the apical tip of the nanopore, on the second side of the nanopore, in a manner such as described with reference to FIG. 2A.

For example, selenium may be expressed co-translationally as selenocysteine (Sec), commonly referred to as the 21^{st} amino acid. Sec is structurally and functionally similar to cysteine (Cys), but differs by a single atom (Se vs S), yet this swap significantly transforms enzyme reactivity in a manner such as described in Hondal et al., “Selenocysteine in thio/disulfide-like exchange reactions,” Antiox Redox Signal 18(13): 1675-1689 (2013), the entire contents of which are incorporated by reference herein. Sec performs dramatically better than Cys, both as a nucleophile in thiol/disulfide-like exchange reactions, and as a leaving group in its regeneration (i.e. higher nucleofugality due to lower pKa and σ*_{S-Se} LUMO energy). Furthermore, Sec is easily expressed in proteins by utilizing defined growth media for E. coli supplemented with Sec, to misload the cysteinyl-tRNA with Sec (i.e. Sec-RNA), in a manner such as described in Liu et al., “Site-specific incorporation of selenocysteine using an expanded genetic code and palladium-mediated chemical deprotection,” J. Am. Chem. Soc. 140 (28): 8807-8816 (2018), the entire contents of which are incorporated by reference herein. Other methods such as reprogramming the opal (stop) codon UGA to encode for Sec (via native or non-native biomachinery) may be performed in a manner such as described in Cheng et al., “Overexpression of recombinant selenoproteins in E. Coli,” Chavatte L. (eds) Selenoproteins. Methods in Molecular Biology, vol 1661, Humana Press, New York, NY, https://doi.org/10.1007/978-1-4939-7258-6_17, pages 231-240 (2017), the entire contents of which are incorporated by reference herein. Sec alternatively may be expressed in minimal media with all the sulfur swapped for selenium in a manner such as described in Schaefer et al., “⁷⁷Se enrichment of proteins expands the biological NMR toolbox,” Journal of Molecular Biology 425(2): 222-231 (2013), the entire contents of which are incorporated by reference herein. For details regarding MspA mutations suitable for use in include SeC in MspA, see Cao et al., “Giant single molecule chemistry events observed from a tetrachloroaurate (III) embedded Mycobacterium smegmatis porin A nanopore,” Nat Commun. 10(1): 5668 (2019), the entire contents of which are incorporated by reference herein. For details regarding Sec incorporation, see Thyer et al., “Engineered rRNA enhances the efficiency of selenocysteine incorporation during translation,” Journal Am Chem Soc. 135 (1): 2-5 (2013), the entire contents of which are incorporated by reference herein.

In some examples, reaction between the trigger (dithiol bond in PIT) and the Sec group coupled to the nanopore may oxidize the Sec group to the selenosulfide form. The Sec group then may be regenerated by reducing the selenosulfide form using a suitable reducing agent or combination of reducing agents in fluid 120′, for example using glutathione or selenoenzyme thioredoxin. The reducing agent may be compartmentalized on the second side of the nanopore, e.g., in a manner such as described elsewhere herein. For example, the reducing agent may be attached to a particle. The regenerated Sec group then may be used as an initiator for another PIT, e.g., a PIT of a 3′-blocking group of a subsequent nucleotide being added to the 3′ end 153 of duplex 154.

It will be appreciated that the PIT based 3′-blocking group suitably may be modified so as to improve solubility, stability, and/or deblocking kinetics. For example, the 2-mercaptopyridine optionally may by modified to include one or more electron-withdrawing groups (e.g., —CF₃, —NO₂, —CO₂R (where R is any hydrocarbon group, halide, or the like), which may increase deblocking kinetics. Additionally, or alternatively, spacer X in Formula 1 may include one or more additional intermediary linkers, such dicarbamates, to tune reactivity and/or stability. Still other options readily may be envisioned based on the present teachings. Additionally, or alternatively, the PIT may be extended by one or more disulfides in a manner similar to that described with reference to SIT.

In some examples of a PIT based 3′-blocking group, 3′-blocking group has the structure:

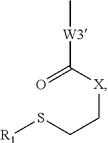

where W is O or NH, X is O or N, and R₁ is selected from the group consisting of

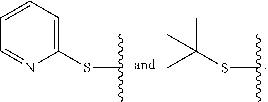

Such a 3′-blocking groups may be degraded using any suitable initiator or combination of initiators, illustratively a selenocysteine group coupled to the second side of a nanopore.

Still other types of 3′-blocking groups may be used, some of which may be encompassed within Formula I. In some examples, 3′-blocking group may include an elongated body that may be degraded via cyclization(s) of the monomer(s) Yₙ. Accordingly, the monomer(s) may be configured so as respectively to cyclize responsive to activation of a trigger, e.g., activation of a chemical entity that initiates cyclization of at least one monomer Y. In examples in which n is two or more, the cyclization of a first one of the monomers may initiate cyclization of a second one of the monomers, and so on, in a cascading cyclization process. Such initiation of cyclization of a given one of the monomers, responsive to cyclization of a prior one of the monomers, may continue until all of the monomers are cyclized. Illustratively, the monomer(s) Yₙ may include:

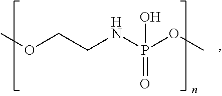

where n is one or more. In other examples, the monomer(s) Yₙ may include:

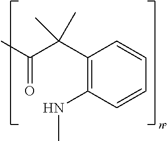

where n is one or more. In still other examples, the monomer(s) Yₙ may include:

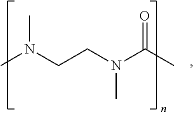

where n is one or more.

In other examples, the elongated body of 3′-blocking group may be degraded via self-immolation of the monomer(s) Yₙ. Accordingly, the monomer(s) may be configured so as respectively to self-immolate responsive to activation of a trigger, e.g., activation of a chemical entity that initiates self-immolation of at least one monomer Y. In examples in which n is two or more, the self-immolation of a given one of the monomers may initiate self-immolation of a second one of the monomers Y, and so on. Such initiation of self-immolation of a given one of the monomers, responsive to self-immolation of a prior one of the monomers, may continue until all of the monomers have self-immolated. Illustratively, the monomer(s) Yₙ may include:

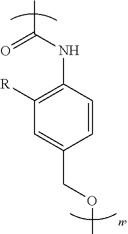

where n is one or more, and in which R is, illustratively, H or alkyl. In other examples, the monomer(s) Yₙ may include:

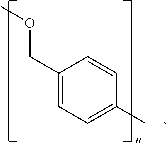

where n is one or more.

It will be appreciated that any suitable value of n may be used in any of the above examples or any other 3′-blocking groups that may be envisioned. For example, n may have any suitable value, e.g., between about 1 and about 100, between about 1 and about 50, between about 1 and about 20, between about 1 and about 10, or between about 1 and about 5. In examples in which 3′-blocking group includes a polymer, n may have any value of two or greater, e.g., between about 2 and about 100, between about 2 and about 50, between about 2 and about 20, between about 2 and about 10, or between about 2 and about 5. In some examples, the components of 3′-blocking group collectively may have a sufficient length that trigger R₁ may be located on second side 112 of nanopore 110, while the nucleotide remains on the first side 111 of nanopore 110. Illustratively, 3′-blocking group may have a length of at least about 2 nm, e.g., between about 2 and about 100 nm, between about 2 and about 50 nm, between about 2 and about 20 nm, between about 2 and about 10 nm, or between about 2 and about 5 nm. However, as noted elsewhere herein, the present 3′-blocking groups are not limited to use with a nanopore, and as such may have any suitable length, e.g., need not necessarily have a sufficient length to locate the trigger on the second side of a nanopore, but may have a sufficient length to interact with an initiator located on the second side of the nanopore.

Any suitable trigger R₁ may be used to initiate degradation of 3′-blocking group. For example, responsive to activation, the trigger may cause cyclization of a monomer Y which, in examples in which n is two or more, may cause cyclization of another monomer Y, and so on, e.g., until all of the monomers are cyclized. Or, for example, responsive to activation, the trigger may cause self-immolation of a monomer Y which, in examples in which n is two or more, may cause self-immolation of another monomer Y, and so on, e.g., until all of the monomers are self-immolated. The degradation may replace X (if included) with H, thus replacing 3′-blocking group with a 3′-OH group or 3′-NH₂ group. Alternatively, the degradation may replace that Y with H, thus replacing 3′-blocking group with a 3′-OH group or 3′-NH₂ group. It will be appreciated that the present triggers may be used with 3′-blocking groups that include any suitable number of monomers, and that may be degraded using any suitable process(es).

Some triggers R₁, when activated, form primary amines that degrade the elongated body of 3′-blocking group. Illustratively, the trigger R₁ may include an azide. The initiator may reduce the azide to a primary amine that degrades the elongated body. The azide may be located at the second end of the elongated body (that is, Z may not be included). Alternatively, the azide may be located along the elongated body, between the first end and the second end (that is, Z may be included). Example initiators for use in reducing an azide to a primary amine that degrades the elongated body and that may be substantially on the second side of the nanopore include, but are not limited to, polymer-bound triphenylphosphine, polymer-bound phenyldi(o-tolyl)phosphine, or polymer-bound tris(hydroxypropyl)phosphine (THP), although other such initiators readily may be envisioned.

In other examples, the trigger R₁ may include a secondary amine. The initiator may convert the secondary amine to a primary amine that degrades the elongated body. The secondary amine may be located at the second end of the elongated body (that is, Z may not be included). Alternatively, the secondary amine may be located along the elongated body, between the first end and the second end (that is, Z may be included). Initiators readily may be selected that convert secondary amines into primary amines and that may be coupled to particles or otherwise substantially retained on the second side of the nanopore. In some examples, the secondary amine includes:

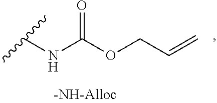 [SMILES: *NC(=O)OCC=C]

in which Alloc refers to allyloxycarbonyl, and for which an example initiator includes a polymer-bound Pd⁰-phosphine complex. In other examples, the secondary amine includes:

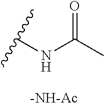 [SMILES: *NC(C)=O]

for which an example initiator includes an acylase enzyme (example biological entity) which may be sufficiently large to be substantially retained on the second side of the nanopore. In still other examples, the secondary amine includes:

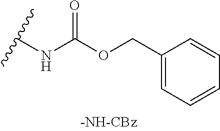 [SMILES: *NC(=O)OCc1ccccc1]

for which an example initiator includes palladium on activated carbon (Pd—C) and H₂. In yet other examples, the secondary amine includes:

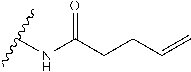 [SMILES: *NC(=O)CCC=C]

for which an example initiator includes particle bound N,N′-dibromodimethylhydantoin (DBDMH). For further details regarding use of DBDMH to reduce such a secondary amine, see Limanto et al., “An efficient chemoenzymatic approach to (S)-γ-fluoroleucine ethyl ester,” J. Org. Chem. 70:2372-2375 (2005), the entire contents of which are incorporated by reference herein.

In other examples, the trigger R₁ may include a nitro group (—NO₂). The nitro group may be located at the second end of the elongated body (that is, Z may not be included). Alternatively, the nitro group may be located along the elongated body, between the first end and the second end (that is, Z may be included). The initiator may convert the —NO₂ to a primary amine that degrades the elongated body. Any suitable initiator may be used to perform such conversion, such as a palladium catalyst which is particle bound. Example palladium catalysts for reducing a nitro group to a primary amine are disclosed in the following references, the entire contents of each of which are incorporated by reference herein: Mase et al., “Fine-bubble-based strategy for the palladium-catalyzed hydrogenation of nitro groups: Measurement of ultrafine bubbles in organic solvents,” Synlett 28:2184-2188 (2017); and Rahaim et al., “Pd-catalyzed silicon hydride reductions of aromatic and aliphatic nitro groups,” Org. Lett. 7 (22): 5087-5090 (2005). In still other examples, the initiator for reducing the nitro group may include a nitroreductase enzyme such as described in Saneyoshi et al., “Bioreductive deprotection of 4-nitrobenzyl group on thymine base in oligonucleotides for the activation of duplex formation,” Bioorganic & Medicinal Chemistry Letters 25:5632-5635 (2015), the entire contents of which are incorporated by reference herein.

While some examples, such as described above, may use an initiator to convert a trigger R₁ into a primary amine (activated trigger) that degrades the elongated body, it will be appreciated that still other types of triggers R₁ (and activated triggers) may be used. For example, the trigger may include:

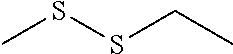

The initiator may convert the trigger to a thiol that degrades the elongated body. A nonlimiting example of such an initiator includes a particle-bound phosphine such as described with reference to FIGS. 2C and 4A-4B. The trigger may be located at the second end of the elongated body (that is, Z may not be included). Alternatively, the trigger may be located along the elongated body, between the first end and the second end (that is, Z may be included).

In still other examples, the trigger R₁ may include allyloxymethoxy (AOM):

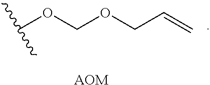

The initiator may convert the AOM to an alcohol (activated trigger) that degrades the elongated body. Nonlimiting examples of such an initiator include Pd⁰-phosphine complexes, e.g., such as described in U.S. Patent Publication No. 2021/0403500, filed on Jun. 21, 2021 and entitled “Nucleosides and Nucleotides with 3′ Acetal Blocking Group;” Peterson et al., “Controlled depolymerization: Stimuli-responsive self-immolative polymers,” Macromolecules 45 (18): 7317-7328 (2012); and Kevwitch et al., “Vanillin and o-vanillin oligomers as models for dendrimer disassembly,” New J. Chem. 36:492-505 (2012), the entire contents of each of which are incorporated by reference herein. The Pd⁰-phosphine complex may be coupled to a particle or otherwise retained on the second side of the nanopore. The trigger may be located at the second end of the elongated body (that is, Z may not be included). Alternatively, the trigger may be located along the elongated body, between the first end and the second end (that is, Z may be included).

In yet other examples, the trigger R₁ may include:

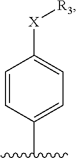

where X is O or NH, and wherein R₃ is H or a protecting group if X is O, and wherein R₃ is H or alkyl if X is NH. The initiator may convert such a trigger to:

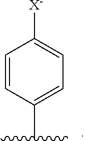

A nonlimiting example of such an initiator includes a biological entity such as a protease enzyme, or a redox chemical moiety. Example enzymes (e.g., plasmins or amidases) or redox chemical moieties (such as Zn/AcOH) that may be used as initiators are described in the following references, the entire contents of each of which are incorporated by reference herein: Peterson et al., “Controlled depolymerization: Stimuli-responsive self-immolative polymers,” Macromolecules 45 (18): 7317-7328 (2012); Weinstain et al., “Self-immolative comb-polymers: multiple-release of side-reporters by a single stimulus event,” Chemistry 14 (23): 6857-6861 (2008); Weinstain et al., “Activity-linked labeling of enzymes by self-immolative polymers,” Bioconjugate Chem. 20 (9): 1783-1791 (2009); de Groot et al., “Elongated multiple electronic cascade and cyclization spacer systems in activatable anticancer prodrugs for enhanced drug release,” J. Org. Chem. 66:8815-8830 (2001); and Warnecke et al., “2,4-Bis(hydroxymethyl) aniline as a building block for oligomers with self-eliminating and multiple release properties,” J. Org. Chem. 73 (4): 1546-1552 (2008). The trigger may be located at the second end of the elongated body (that is, Z may not be included). Alternatively, the trigger may be located along the elongated body, between the first end and the second end (that is, Z may be included).

Purely for purposes of illustration, some specific, nonlimiting example combinations of the foregoing monomers, triggers, and initiators now will be described. Other combinations readily may be envisioned based on the present teachings.

In some examples, 3′-blocking group includes a SIT of Formula I in which the monomer Yₙ may include:

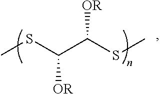

X is carbonate or carbamate coupled to a mercaptoethanol, n is at least two, R₁ includes 2-mercaptopyridine, and X and Z are not included. The initiator converts the trigger to a thiolate group which degrades 3′-blocking group. In some examples, 3′-blocking group includes a PIT having the structure:

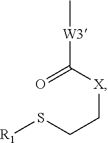

in which X is O or NH and the trigger includes 2-mercaptopyridine. The initiator converts the trigger to a thiolate group which degrades 3′-blocking group using the following cascading cyclization scheme:

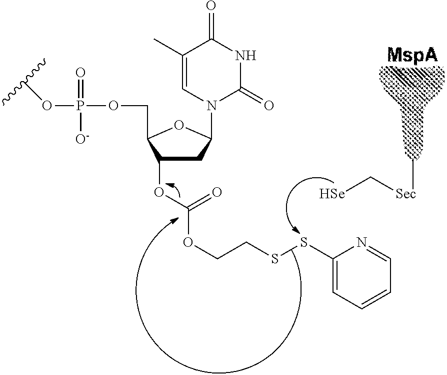

In other examples, the monomer Yₙ may include:

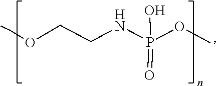

X and Z are not included, and the initiator converts the trigger R₁ to a primary amine which degrades the elongated body of 3′-blocking group using the following cascading cyclization scheme:

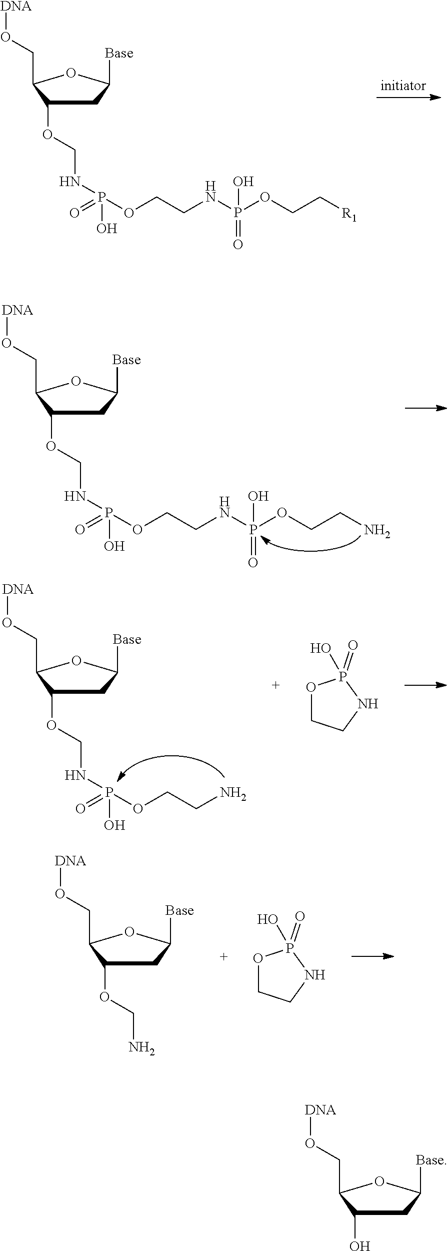

While n=2 for the above scheme, it will be appreciated that 3′-blocking group readily may be modified so as to include any suitable value of n.

A first set of example options for trigger R₁, and corresponding example initiators including biological or chemical entities, for the above scheme, are provided below:

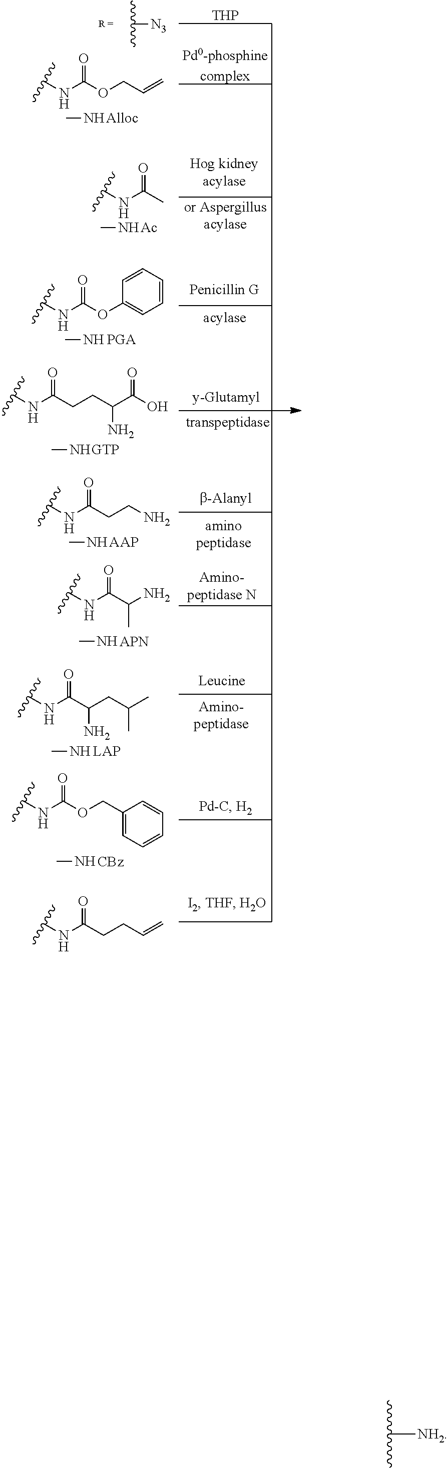

Other options for removing 3′-blocking groups that include peptide bonds, such as illustrated above, include Penicillin G acylase (PGA), γ-Glutamyltranspeptidase (GTP), β-Alanyl aminopeptidase (AAP), Aminopeptidase N (APN), and Leucine Aminopeptidase (LAP).

In certain other examples, the monomer Yₙ may include:

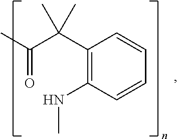

X is —CH₂—NH—, Z is not included, and the initiator converts the trigger R₁ to a primary amine which degrades the elongated body of 3′-blocking group using the following cascading cyclization scheme in which a single cyclization is shown for simplicity:

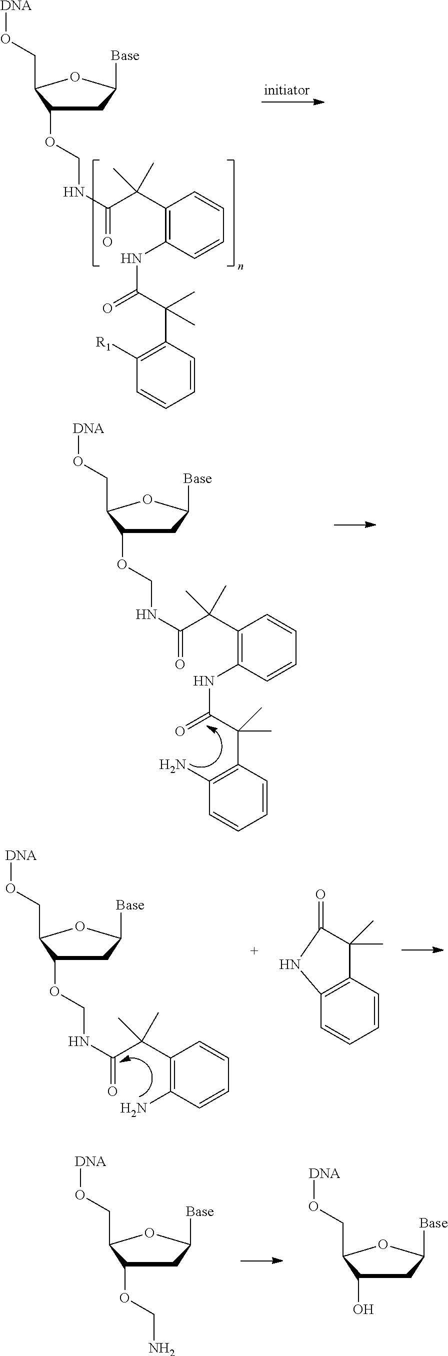

It will be appreciated that 3′-blocking group readily may be modified so as to include any suitable value of n.

A set of example options for trigger R₁, and corresponding example initiators, for the above scheme, are provided below:

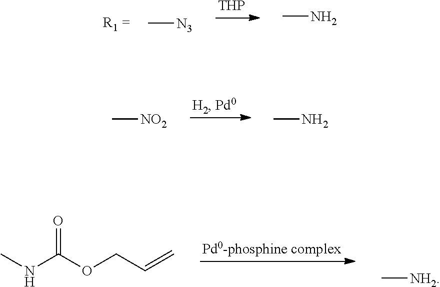

In still other examples, Z is included, and may provide the second end of 3′-blocking group. Z may, in some examples, be used to bring the initiator sufficiently into proximity of the trigger as to be able to react with the trigger. Illustratively, Z may include a target, and the target may be bound by a protein that includes the initiator. For example, FIGS. 5A-5B schematically illustrate additional compositions and operations for deblocking 3′-blocked nucleotides. In the example illustrated in FIG. 5A, 3′-blocking group 530 coupled to nucleotide 520 includes Yₙ monomers (where n is one or more), trigger R₁ located along the elongated body of 3′-blocking group, and extension Z which includes a target that may be bound by protein 570 to which one or more initiators 535, e.g., a plurality of initiators 535, are coupled. In a manner similar to that described with reference to FIGS. 3A-3C, the base of nucleotide 520 may be located on first side 111 of nanopore 110, while trigger R₁ and protein 570 may be located on second side 112 of nanopore 110 such that initiator(s) 535 may activate trigger R₁. Protein 570 may be sufficiently large as to be unable to pass through aperture 113 of nanopore 110, and thus the initiator(s) 535 coupled thereto may be substantially unable to activate any triggers R₁ that are located on first side 111 of nanopore 110.

It will be appreciated that any suitable targets may be used, and any suitable proteins that may be used to bind to such targets may be used that may be modified so as to include or be coupled to one or more initiators. Similarly, any suitable triggers R₁ may be used that may be activated using such initiator(s) and that may initiate degradation of a suitable 3′-blocking group. Illustratively, target Z illustrated in FIG. 5A may include biotin, and protein 570 may include streptavidin which is modified so as to include a plurality of initiators 535, such as a phosphine (—PR′₂), where R′ may be selected from the group consisting of:

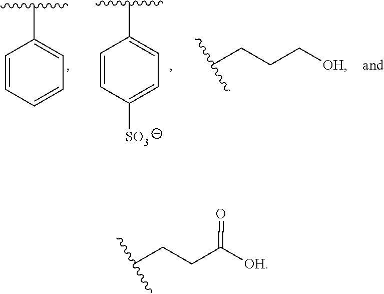

It will be appreciated that any other initiators, such as the non-limiting examples provided herein, instead may be coupled to protein 570. Trigger R₁ may include, for example, an azide or a disulfide such as

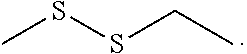

The phosphine, or other suitable initiator, may convert the azide to a primary amine that degrades the elongated body, or may convert the disulfide to a thiol that degrades the elongated body.

In one nonlimiting example, nucleotide 520 coupled to 3′-blocking group 230 may have the structure:

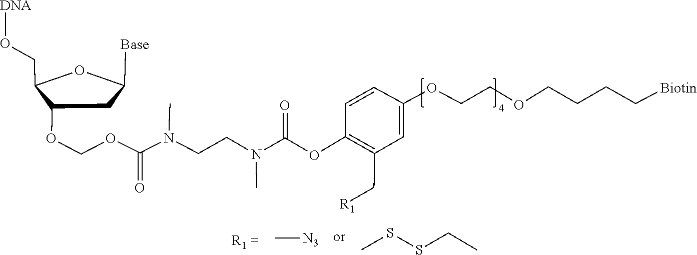

The base of the nucleotide 520 may be on first side of 111 nanopore 110, while trigger R₁ and initiator 535 (e.g., phosphine coupled to protein 570) may be located on second side 112 of the nanopore. Protein 570 may bind the biotin on the second side of nanopore 110, following which initiator 535 may react with trigger R₁ to generate activated trigger R₁′H which may initiate degradation of the elongated body using the following cyclization scheme:

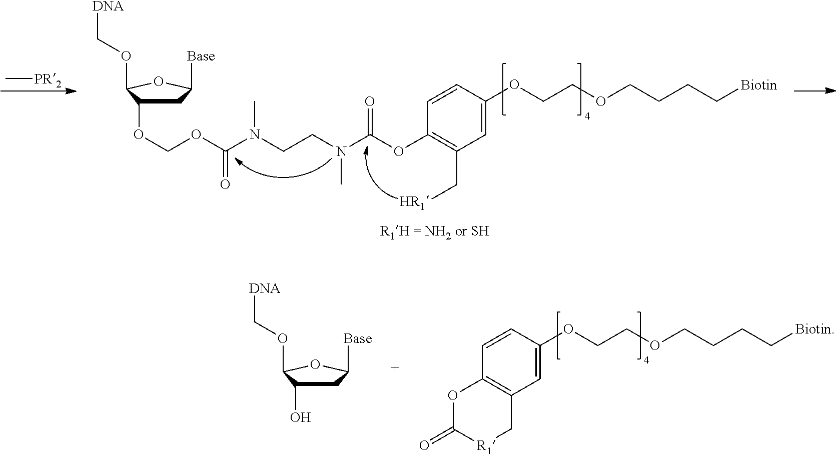

In some examples, the present 3′-blocking groups may be degraded using self-immolation. Some examples of self-immolating 3′-blocking groups are described with reference to SIT and PIT. Another example trigger that may be activated to initiate self-immolation is:

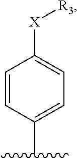

where X is O or NH, and wherein R₃ is H or a protecting group if X is O, and wherein R₃ is H or alkyl if X is NH. Nonlimiting examples of monomers Yₙ that may be used with such a trigger include, but are not limited to —[O—CH₂]ₙ—, —[O—CH₂—O]ₙ—, and —[O—CHO—O]ₙ—. In this regard, the trigger may be considered a benzyloxymethyl group. For nonlimiting examples of benzylmethoxy groups, see Saneyoshi et al., “Development of bioreduction labile protecting groups for the 2′-hydroxyl group of RNA,” Org. Lett. 22 (15): 6006-6009 (2020), the entire contents of which are incorporated by reference herein. The benzyloxymethyl group optionally may be substituted. For example, the nucleotide having the 3′-blocking group may have a structure selected from the group consisting of:

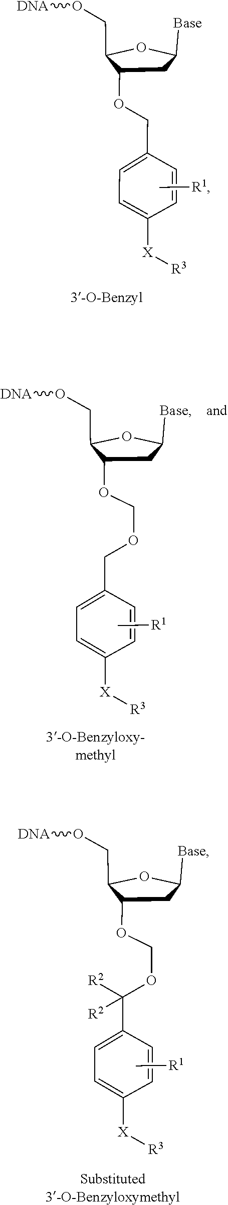

where R₁ may be alkyl, aryl, alkoxyl, aryloxy, aminoalkyl, aminoaryl, or halogenyl and R₂ may be alkyl, aryl, or halogenyl.

Illustratively, an example scheme in which activation of such a trigger results in self-immolation of a 3′-blocking group, is below:

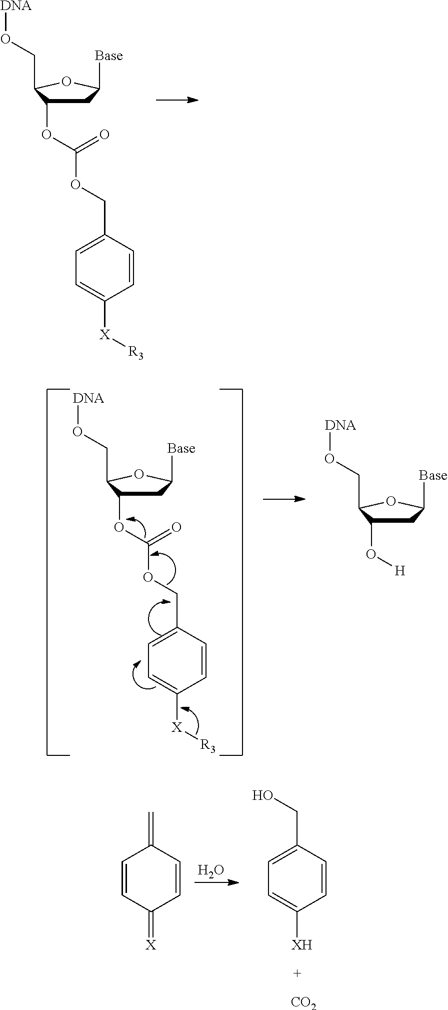

Another example scheme in which activation of such a trigger results in self-immolation of a 3′-blocking group, is below:

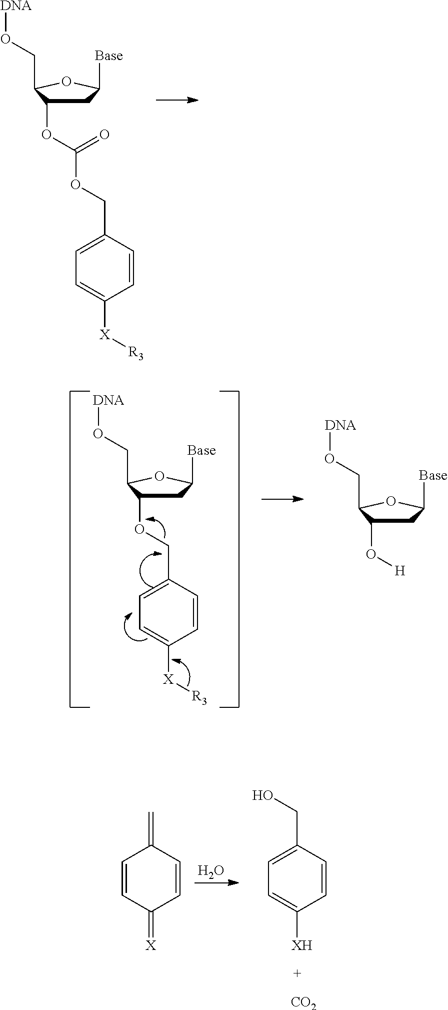

Another example scheme in which activation of such a trigger results in self-immolation of a 3′ blocking group, is below:

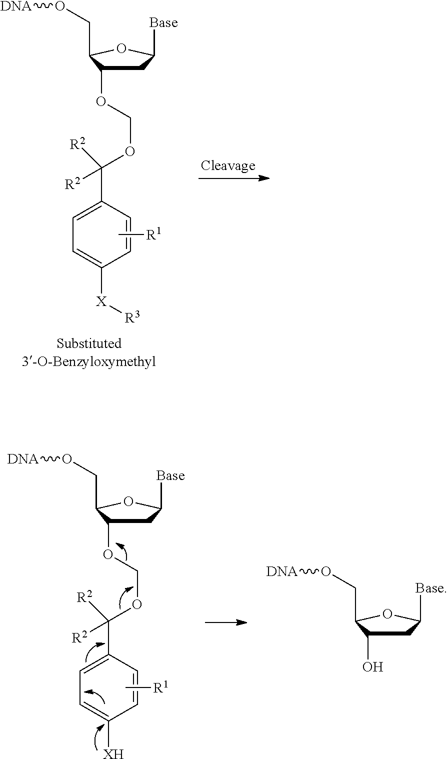

Yet another example scheme in which activation of such a trigger results in self-immolation of a 3′-blocking group is below:

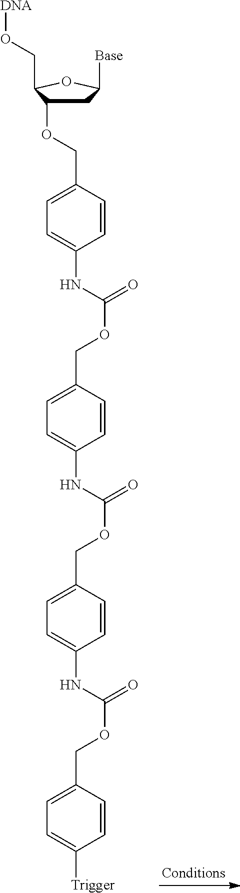

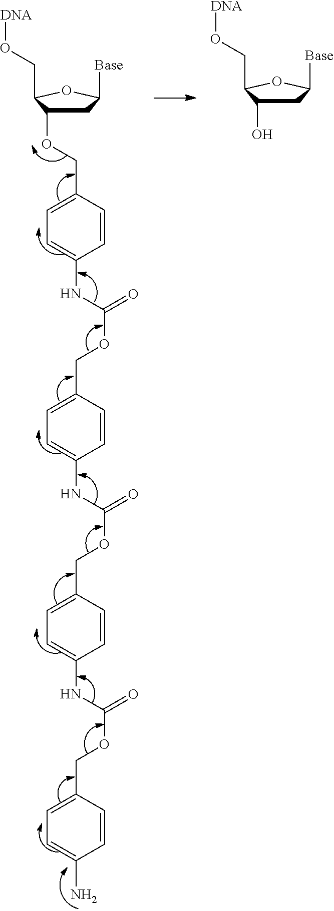

in which Yₙ is:

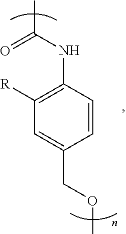

n is 4, R is H, and X and Z are not included.

Yet another example scheme in which activation of such a trigger results in self-immolation of a 3′-blocking group is below:

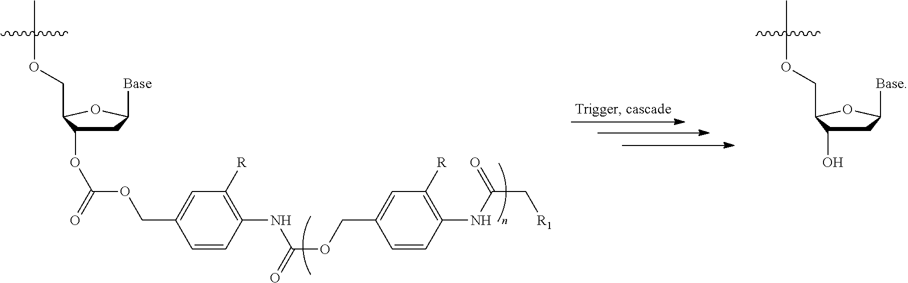

The 3′-blocking group in the two preceding schemes may be prepared in any suitable manner, e.g., using a scheme such as illustrated below:

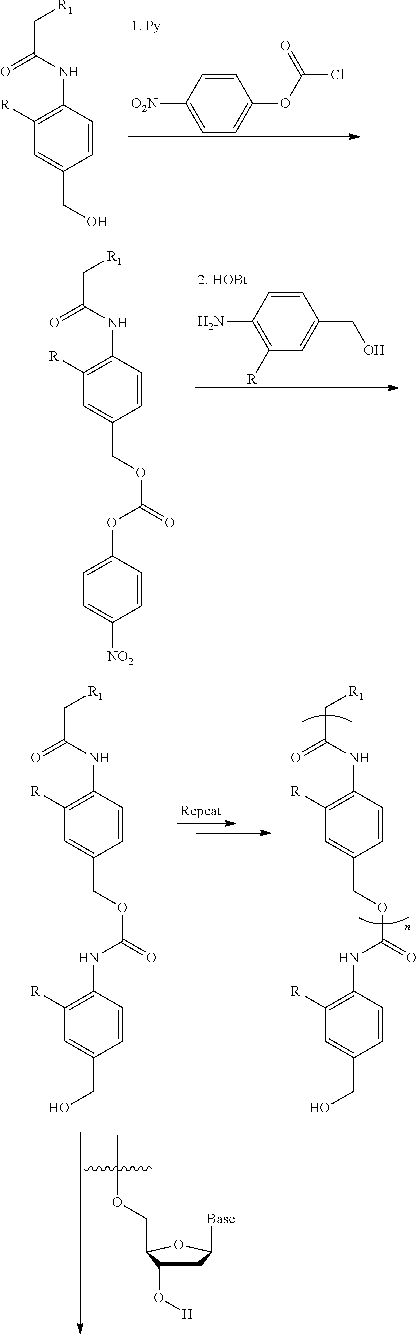

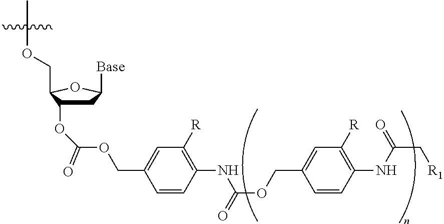

It will be appreciated that any suitable combination of the triggers and initiators provided herein may be used to degrade the present 3′-blocking groups. For example, a 3′-blocking group may include two or more different types of monomers (that is, not all Y need be the same as one another in the blocking group coupled to a given nucleotide). Activation of the trigger may initiate degradation of a first type of monomer, and the degradation of that type of repeating unit may initiate degradation of a second, different type of monomer.

Still other examples of 3′ blocking groups include self-immolative carbonate and carbamates. For example, carbonates may be useful at a pH of about 7 or below, while carbamates may be useful at a pH of about 7 or higher. Nonlimiting examples of nucleotides coupled to self-immolative carbonates useful at pH of about 7 or below include:

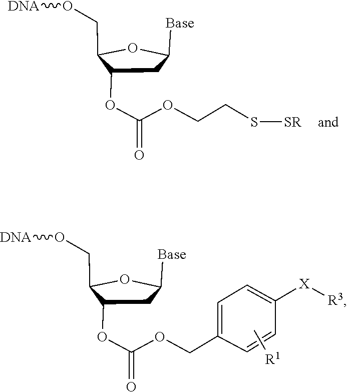

where R₁ and R₃ are as defined elsewhere for benzyloxymethyl trigger groups.

A nonlimiting example of a nucleotide coupled to a self-immolating carbamate at pH of about 7 or below is:

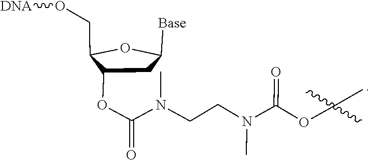

A nonlimiting example of a nucleotide coupled to a self-immolating carbamate at pH of above about 7 is:

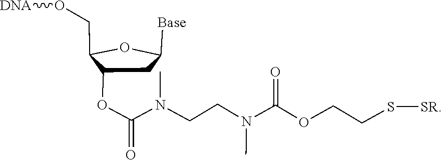

An example scheme for removing such self-immolating carbamate from the nucleotide is shown below:

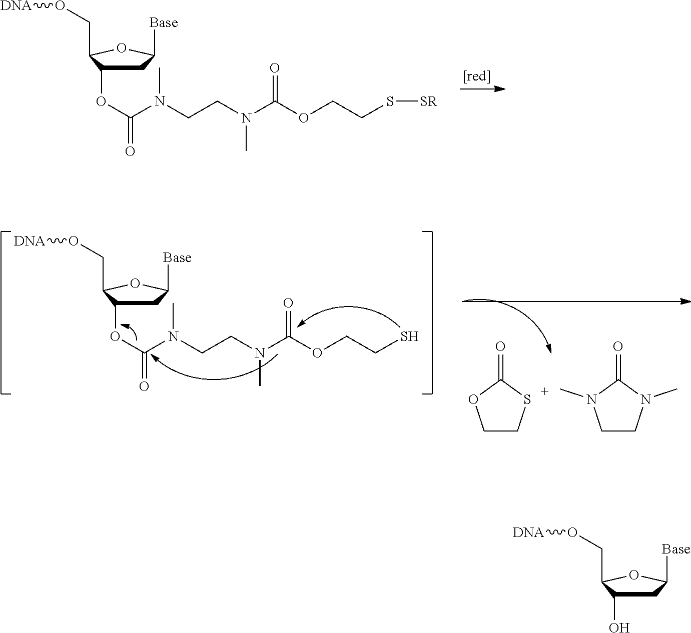

Another nonlimiting example of a nucleotide coupled to a self-immolating carbamate at pH of above about 7 is:

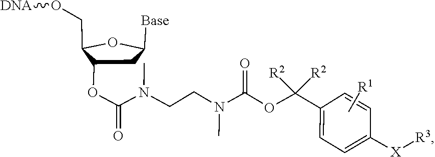

where R₁, R₂, and R₃ are as defined elsewhere for benzyloxymethyl trigger groups. An example scheme for removing such self-immolating carbamate from the nucleotide is shown below:

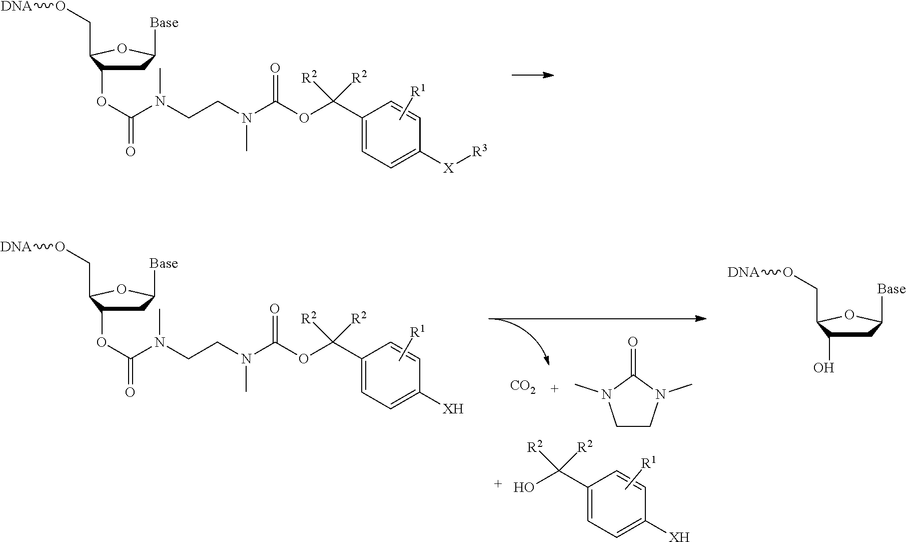

In some examples herein, 3′-blocked nucleotides may be deblocked using suitable reductive or oxidative conditions. Examples of such dithiane or 4-nitrobenzyloxymethyl groups are shown below:

### a) Dithiane Block Removed Under Oxidative Conditions Through β-Elimination

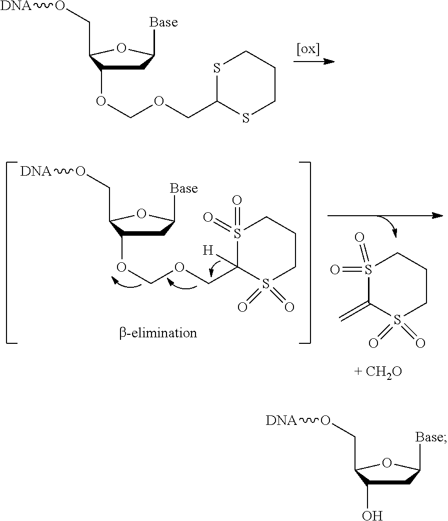

or

### B) Substituted 4-Nitrobenzyloxymethyl Block Removed Under Reducing Conditions

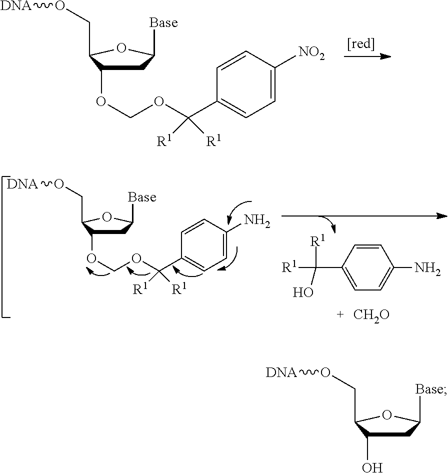

Another example redox system is based on a quinone-hydroquinone redox system and trimethyl lock linker, e.g., as illustrated in the schemes below:

a) Substituted Benzyloxymethyl/Trimehyl Lock Linkers with Quinone-Hydroquinone Trigger

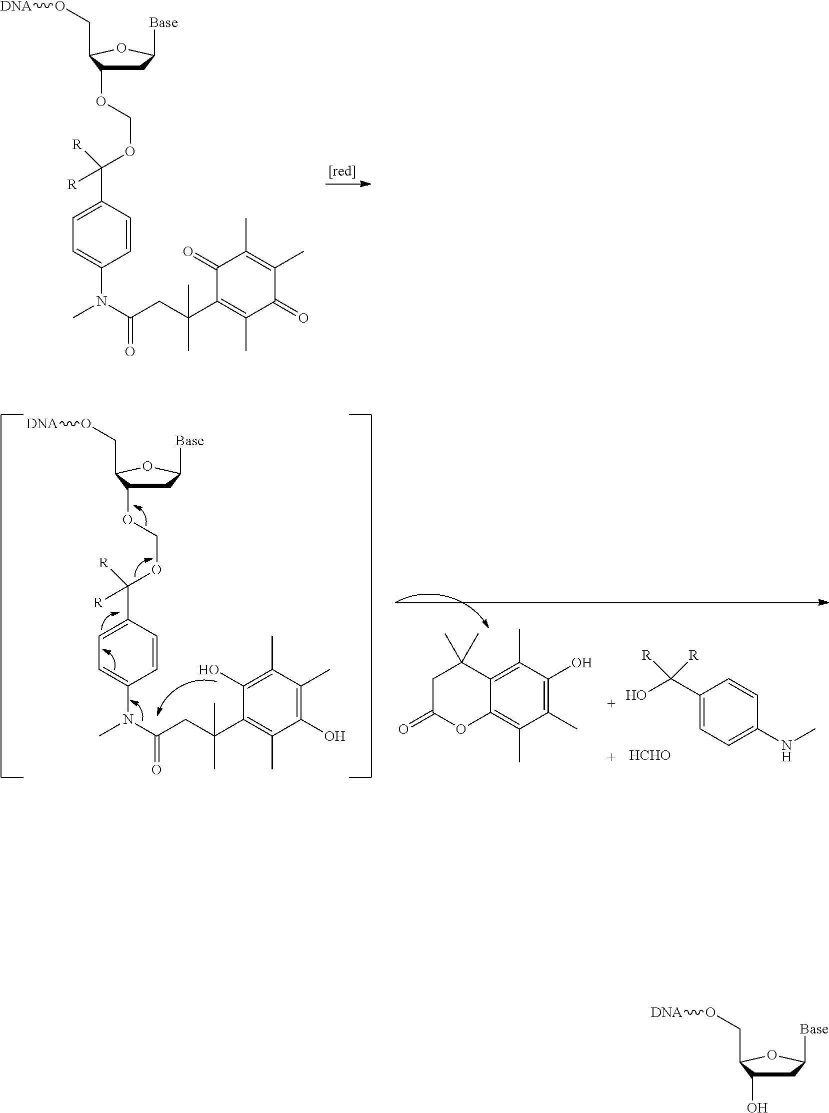

B) Carbamate Linker with Quinone-Hydroquinone Trigger

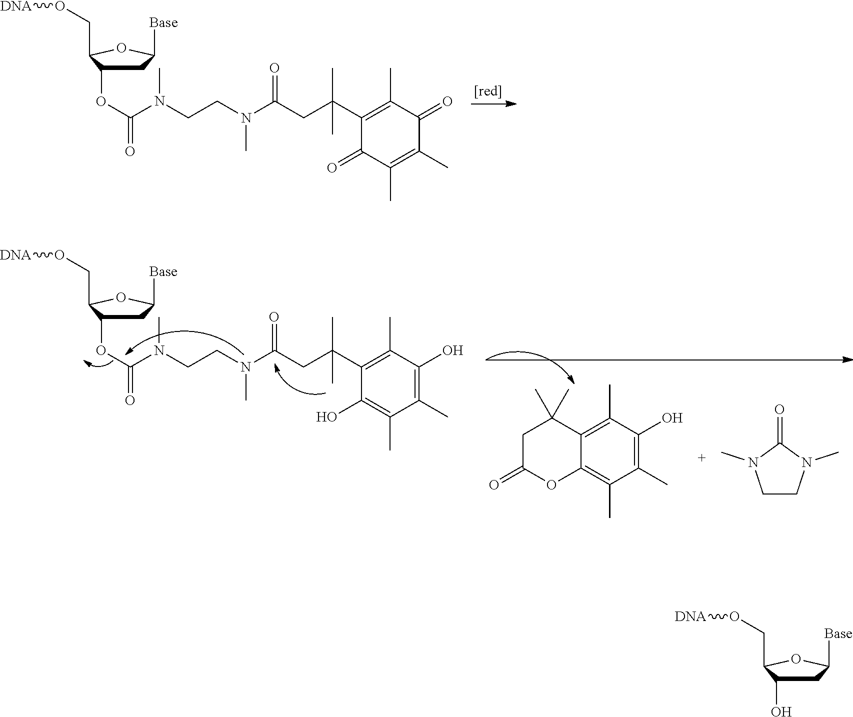

It will be appreciated that compositions and operations such as described with reference to FIGS. 1A-1D, 2A-2C, 3A-3C, 4A-4D, and 5A-5B suitably may be adapted so as couple a nucleotide to a 3′-blocking group, and to controllably deblock such nucleotide. For example, FIG. 6 illustrates a flow of operations in an example method for deblocking 3′-blocked nucleotides. Method 600 illustrated in FIG. 6 includes disposing a nucleotide within an aperture of a nanopore on a first side of the nanopore (operation 610). The nucleotide may be coupled to a 3′-blocking group including a trigger. In nonlimiting examples, 3′-blocking group may include an elongated body including a first end, a second end, and the trigger. The nucleotide and the first end may be located on the first side of the nanopore. For example, nucleotide 121 may be disposed within aperture 113 of nanopore 110 in a manner such as described with reference to FIGS. 1B, 2B, and FIG. 3A. Such nucleotide may be located on first side 111 of the nanopore while trigger 134 coupled thereto may be in sufficient proximity to the second side 112 of the nanopore in a manner such as described with reference to FIG. 1B, or may be in sufficient proximity to an initiator within the nanopore aperture in a manner such as described with reference to FIG. 2B, or may be located on second side 112 of the nanopore in a manner such as described with reference to FIG. 3A.

Method 600 also may include selectively activating the trigger (operation 620). In some examples, the initiator may be is located on the second side of the nanopore and substantially not located on the first side of the nanopore. For example, initiator 135 may be located on second side 112 of nanopore 110 and substantially not located on first side 111 of the nanopore, and may activate trigger 134 or trigger 334 in a manner such as described with reference to FIG. 1C or FIG. 3B. In other examples, initiator 135 may be located within the aperture 113 of the nanopore 110, and may activate trigger 134 in a manner such as described with reference to FIG. 2C. Method 600 illustrated in FIG. 6 further may include using the activated trigger to remove 3′-blocking group from the nucleotide (operation 630). Removing 3′-blocking group may provide the nucleotide with a 3′-OH group, or with a 3′-NH₂ group. In nonlimiting examples such as described with reference to FIGS. 3A-3C, responsive to activation of trigger 135, elongated body 131 may degrade, e.g., via cascading cyclization. Example triggers, initiators, and elongated bodies are described elsewhere herein.

### Methods of Synthesizing Polynucleotides

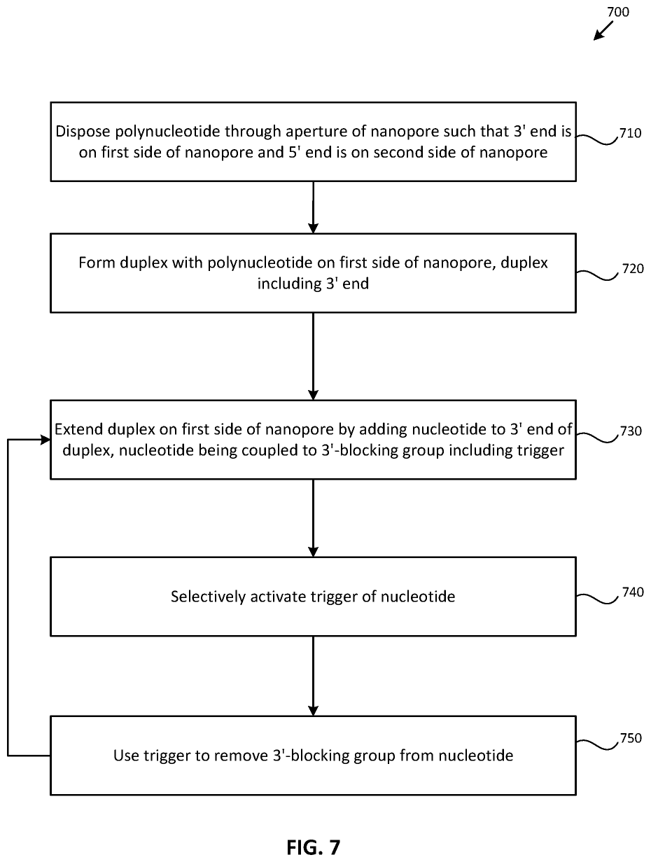

It will further be appreciated that compositions and operations such as described with reference to FIGS. 1A-1D, 2A-2C, 3A-3C, 4A-4D, 5A-5B, and 6 suitably may be adapted for use in various methods of synthesizing polynucleotides, including but not limited to sequencing-by-synthesis (SBS), but may be used in any suitable application or context for which it is desirable to use 3′-blocked nucleotides and then deblock such nucleotides. For example, FIG. 7 illustrates a flow of operations in an example method for synthesizing a polynucleotide using 3′-blocked nucleotides. Method 700 illustrated in FIG. 7 may be performed using a nanopore comprising a first side, a second side, and an aperture extending through the first and second sides. Method 700 may include disposing a polynucleotide through the aperture of a nanopore such that a 3′ end of the second polynucleotide is on the first side of the nanopore, and a 5′ end of the second polynucleotide is on the second side of the nanopore (operation 710). Method 700 may include forming a duplex with the polynucleotide on the first side of the nanopore, the duplex including a 3′ end (operation 710). For example, a duplex may be formed by hybridizing nucleotide 140 to nucleotide 150 on first side 111 of nanopore 110 in a manner such as described with reference to FIG. 1A.

Method 700 may include extending the duplex on the first side of the nanopore by adding a nucleotide to the 3′ end of the duplex, the nucleotide being coupled to a 3′-blocking group comprising a trigger (operation 730). For example, the duplex may be contacted with a polymerase 105 and a nucleotide coupled to 3′-blocking group 130 or 330 in a manner such as described with reference to FIG. 1A, 2A, or FIG. 3A. Polymerase 105 may perform such duplex extension by adding 3′-blocked nucleotide to polynucleotide 140 based on the sequence of polynucleotide 150. Method 700 further may include selectively activating the trigger. In some examples, the trigger may be activated using an initiator that is located on the second side of the nanopore and substantially not located on the first side of the nanopore. Illustratively, initiator 135 may be located on second side 112 of nanopore 110 and substantially not located on first side 111 of the nanopore, and may activate trigger 134 or trigger 334 in a manner such as described with reference to FIG. 1C or FIG. 3B. In nonlimiting examples, while nucleotide 121 and first end 132 remain on first side 111 of nanopore 110, trigger 134 may be moved to second side 112 of nanopore 110. Such movement may be induced using any suitable force, such as a bias voltage that circuitry 160 applies between electrodes 102 and 103. Alternatively, initiator 135 may be located within the aperture of the nanopore, and may activate trigger 134 in a manner such as described with reference to FIG. 2C. Method 700 further may include using the activated trigger to remove the 3′-blocking group from the nucleotide (operation 750). In nonlimiting examples, the activated trigger may cause degradation of elongated body 331 coupled to the nucleotide in a manner such as described with reference to FIGS. 3B-3C. Removing 3′-blocking group may provide the nucleotide with a 3′-OH group, or with a 3′-NH₂ group. In nonlimiting examples such as described with reference to FIGS. 3A-3C, responsive to activation of trigger 135, elongated body 131 may degrade, e.g., via cascading cyclization. Example triggers, initiators, and elongated bodies are described elsewhere herein. Optionally, method 700 may include repeating operations 730 through 750 to further extend the duplex by a plurality of additional nucleotides.

### WORKING EXAMPLES

The following examples are intended to be purely illustrative, and not limiting.

Experiments were carried out to examine the feasibility of utilizing a solid supported Pd catalyst in an allylation reaction. O-allylic dichlorofluorescein has low fluorescence quantum yield. Upon cleavage of its O-allyl bond (by an appropriate Pd catalyst), however, it furnishes an anionic fluorescein product which exhibits high levels of fluorescence. As such, this reaction serves as good means by which to measure the activity of a Pd compound.

As Table 2 illustrates, in the presence of a commercially available heterogeneous Pd catalyst (Pd(II) EnCat 30), a significant increase in relative fluorescence units (RFU) was observed after 10 min of reaction, which was improved by heating the reaction at a mild temperature of 55C; similar to typical temperature ranges employed during nucleotide incorporation reactions by DNA polymerase. Conversely, in the absence of any Pd catalyst, there remained only a minor background RFU measured even after 30 min, indicating that essentially no O-allyl cleavage had taken place.

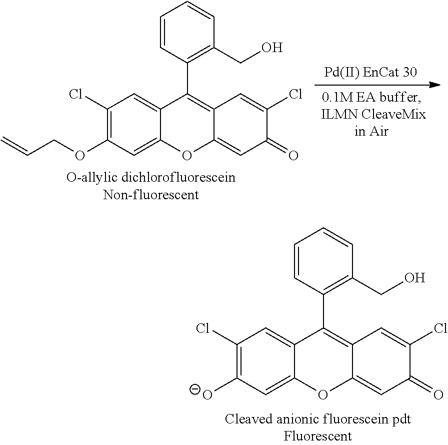 [SMILES: C=CCOc1cc2oc3cc(=O)c(Cl)cc-3c(-c3ccccc3CO)c2cc1Cl ; O=c1cc2oc3cc([O-])c(Cl)cc3c(-c3ccccc3CO)c-2cc1Cl]

TABLE 2
Reaction of a commercially available heterogeneous, solid supported
Pd catalyst (Pd(II) EnCat 30) with O-allylic dichlorofluorescein

| S/N | Conditions | RFU (10 min) |
|---|---|---|
| 1 | 20 C | 4008 |
| 2 | 55 C | 32208 |
| 3 | 55 C (No Pd catalyst added) | 381 |

To further examine the utility of the heterogeneous catalyst, a nucleotide with 3′ AOM blocking group was also studied under similar conditions, in which no specific precautions were taken to maintain an inert environment. As shown in Table 3 below, there was substantially no change observed in the absence of any heterogeneous catalyst. However, up to 37% of the substrate underwent the deprotection reaction in the presence of the heterogeneous catalyst at 55C.

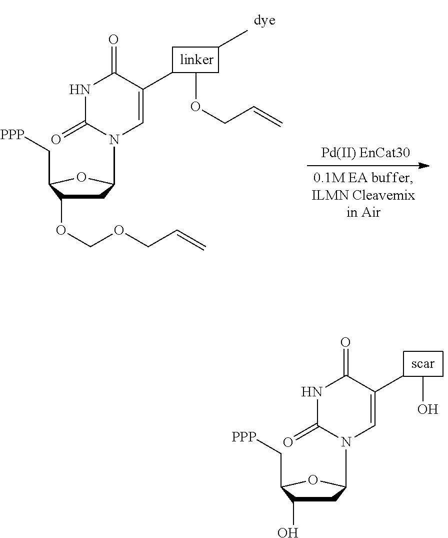

TABLE 3
Reaction of a commercially available heterogenous,
solid supported Pd catalyst (Pd(II) EnCat 30)
with an AOM-blocked nucleotide substrate.

| | | % of 3′-Blocked nucleotides |
|---|---|---|
| | | remaining after 30 min |
| S/N | Conditions | (based on HPLC Peak Area) |
| 1 | 20 C | 92 |
| 2 | 55 C | 63.0 |
| 3 | 55 C (No Pd catalyst added) | 100 |

### Additional Comments

While various illustrative examples are described above, it will be apparent to one skilled in the art that various changes and modifications may be made therein without departing from the invention. The appended claims are intended to cover all such changes and modifications that fall within the true spirit and scope of the invention.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. A method of deblocking a nucleotide using a nanopore comprising a first side and a second side, an aperture extending through the first side and the second side, the method comprising:
disposing the nucleotide within the aperture on the first side of the nanopore, the nucleotide being coupled to a 3′-blocking group comprising a trigger;
selectively activating the trigger using an initiator; and
using the trigger to remove 3′-blocking group from the nucleotide.

2. The method of claim 1, wherein the initiator is located on the second side of the nanopore and substantially not located on the first side of the nanopore.

3. The method of claim 1, wherein the initiator is only located on the second side of the nanopore.

4. The method of claim 1, wherein the initiator is located inside of the aperture.

5. The method of claim 1, wherein the initiator is within a fluid in contact with the second side of the nanopore.

6. The method of claim 1, wherein the initiator is coupled to the second side of the nanopore.

7. (canceled)

8. The method of claim 1, wherein removing 3′-blocking group provides the nucleotide with a 3′-OH group.

9. The method of claim 1, wherein removing the 3′-blocking group provides the nucleotide with a 3′-NH₂ group.

10. The method of claim 1, wherein the initiator comprises a reducing agent.

11. (canceled)

12. The method of claim 1, wherein 3′-blocking group comprises a disulfide bond, and wherein activating the trigger comprises reducing the disulfide bond.

13-16. (canceled)

17. The method of claim 1, wherein the trigger is located on the second side of the nanopore when it is activated.

18. The method of claim 1, wherein 3′-blocking group further comprises an elongated body comprising a first end coupled to the nucleotide, a second end, and the trigger, wherein removing 3′-blocking group comprises degrading the elongated body.

19. (canceled)

20. The method of claim 18, wherein 3′-blocking group comprises one or more monomers, and wherein the elongated body comprises a plurality of the monomers, and wherein degrading the elongated body of 3′-blocking group comprises cascading cyclizations of the monomers.

21. (canceled)

22. The method of claim 20, wherein the one or more monomers are selected from the group consisting of: 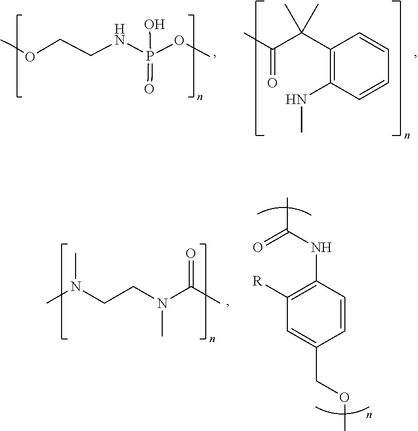
wherein R is H or alkyl, and 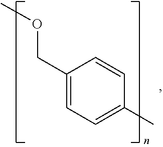
wherein n is 1 or more.

23-51. (canceled)

52. The method of claim 18, wherein the second end comprises a target, the method further comprising binding the target by a protein comprising the initiator.

53-58. (canceled)

59. The method of claim 1, wherein 3′-blocking group is at least about 2 nm long.

60. The method of claim 1, wherein the nanopore comprises a biological nanopore or a solid-state nanopore.

61. (canceled)

62. A method of synthesizing a first polynucleotide using a nanopore comprising a first side, a second side, and an aperture extending through the first side and the second side, the method comprising:
(a) disposing a second polynucleotide through the aperture of the nanopore such that a 3′ end of the second polynucleotide is on the first side of the nanopore, and a 5′ end of the second polynucleotide is on the second side of the nanopore;
(b) forming a duplex with the second polynucleotide on the first side of the nanopore, the duplex comprising a 3′ end;
(c) extending the duplex on the first side of the nanopore by adding a nucleotide to 3′ end of the duplex, the nucleotide being coupled to a 3′-blocking group comprising a trigger;
(d) selectively activating the trigger;
(e) using the trigger to remove 3′-blocking group from the nucleotide; and
(f) repeating operations (c) through (e) to extend the duplex by a plurality of additional nucleotides.

63-65. (canceled)

66. A modified nucleotide, comprising the structure: 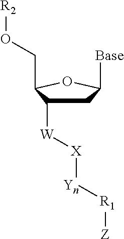
wherein W comprises O or NH₂, X comprises an optional spacer, Y comprises a monomer, n is at least one, Z comprises an optional extension, R₁ comprises a trigger, and R₂ comprises a phosphate or polyphosphate group, and
wherein the trigger is activatable by an initiator so as to degrade Yₙ and X and replace X (if included) or Y with H at 3′ position of the modified nucleotide.

67-76. (canceled)

77. A composition, comprising the modified nucleotide of claim 66 and a nanopore comprising a first side and a second side, wherein the nucleotide is located on the first side of the nanopore and at least R₁ is located on the second side of the nanopore.
